(19) United States
(12) Patent Application Publication
(10) Pub. No.: US 2009/0048199 A1
(43) Pub. Date: Feb. 19, 2009

(54) Hibernation-Related Genes and Proteins, Activators and Inhibitors Thereof and Methods of Use

(75) Inventor: Thomas G. Marr, Denver, CO (US)

Correspondence Address: FULBRIGHT & JAWORSKI L.L.P., 600 CONGRESS AVE., SUITE 2400, AUSTIN, TX, 78701, US

(73) Assignee: Hiberna Corporation, Boulder, CO (US)

(21) Appl. No.: 12/045,116

(22) Filed: Mar. 10, 2008

Related U.S. Application Data
(60) Provisional application No. 60/894,044, filed on Mar. 9, 2007.
(60) Provisional application No. 60/915,773, filed on May 3, 2007.

(51) Int. Cl. A61K 31/711; C12Q 1/68; A61P 9/04; A61P 9/02; A61P 9/10; A61P 9/12; A61P 9/14

(52) U.S. Cl. 514/44; 435/6

(57) ABSTRACT

The present invention concerns methods and compositions for identifying genes and/or proteins involved in hibernation, activators and/or inhibitors of such genes or proteins, and methods of therapeutic use of such activators and/or inhibitors for treatment of a wide variety of diseases and/or medical conditions. In particular embodiments, such hibernation-related genes may include, but are not limited to, Adfp, Atr4, Cact, Myl6, Ca3, Ckm, Rps2, Lgmn, Fabpa, Fabph and Cyp51a1. Compounds that regulate the activities or functions of the Adfp, Atf4, Cact, Myl6, Ca3, Ckm, Rps2, Lgmn, Fabpa, Fabph and Cyp51a1 genes are known in the art and may be used for therapeutic treatment of diseases involving cardiovascular, gastrointestinal, respiratory, neurologic or immunologic function.

### RELATED APPLICATIONS

This application claims the benefit under 35 U.S.C. §119(e) of provisional U.S. patent application Nos. 60/894,044, filed Mar. 9, 2007, and 60/915,773, filed May 3, 2007, each of which is incorporated herein by reference.

### FEDERALLY FUNDED RESEARCH

The studies disclosed herein were supported in part by grants RR-16466-01 from the National Institutes of Health and EPS-0092040 from the National Science Foundation. The U.S. government may have certain rights to practice the subject invention.

### BACKGROUND

1. Field of the Invention

The present invention concerns methods and compositions for identifying genes and/or proteins involved in hibernation, activators and/or inhibitors of such genes or proteins, and methods of therapeutic use of such activators and/or inhibitors for treatment of a wide variety of diseases and/or medical conditions. More particularly, such activators and/or inhibitors may be of therapeutic use for treatment of diseases or conditions including, but not limited to, ischemia, reperfusion injury, myocardial infarction, atherosclerosis, cardiac arrhythmia, tachycardia, bradycardia, hyperthermia, hypothermia, retinopathy, macular degeneration, glaucoma, stroke, obesity, diabetes, lipidemias, hyperlipidemia, hypercholesterolemia, weight loss, cancer, anemia, shock, hypovolemic shock, rheumatoid arthritis, chronic inflammatory disorders, asthma, muscular dystrophy, neurologic disease, Parkinsonism and Alzheimer's disease.

2. Description of Related Art

Mammalian hibernators achieve significant energy savings by actively suppressing their metabolic rate under the extreme environmental conditions. They have evolved remarkable abilities to sustain the respiratory, cardiovascular, immunological, and neurological responses to metabolic suppression during hibernation that would be fatal in other mammalian species. During the 6-9 month long hibernation season, arctic ground squirrels (Spermophilus parryii) enter the state of torpor by lowering their core body temperatures to as low as −2.9° C. (Barnes 1989). However, they spontaneously re-warm to normal body temperature (36-37° C.) every 10-21 days and maintain that temperature for 15-24 hours before slowly re-entering torpor, despite the large energy cost associated with re-warming.

The adaptive significance of these periodic arousals is unknown. Leading hypotheses include that hibernators arouse to sleep and maintain memory and other cognitive functions (Daan et al. 1991, Trachsel et al. 1991) and to replenish gene products (Martin et al. 1993). Unveiling the biochemical processes in the torpor-arousal cycles in hibernators may provide insight into the design of new treatments for human conditions such as stroke, ischemia, and reperfusion (Carey et al. 2003).

It has been proposed that the hibernation phenotype results from the differential expression of existing genes, rather than the creation of novel genes (Srere et al. 1992). Recent large-scale gene expression studies by several groups of investigators on different hibernating mammalian species have provided evidence that global gene expression changes take place at the mRNA level in a tissue-specific manner during hibernation. Brauch et al. (2005) generated a heart-specific cDNA library from thirteen-lined ground squirrels (Spermophilus tridecemlineatus) and examined the differential expression of 48 genes in heart by comparing the mRNA profiles in winter torpid with summer active ground squirrels. Using the microarrays generated from the cDNA library in golden-mantled ground squirrel (Spermophilus lateralis), Williams et al. (2005) examined differential expression between winter torpid and summer active ground squirrels in 102 cDNAs in liver, 115 cDNAs in heart, and 78 cDNAs in brain respectively. Although they included animals in arousal state in their study, they did not find any significant gene differential expression between animals in torpor and arousal states in any of three tissues.

Previously, we examined the differential expression of 625 genes in brown adipose tissue (BAT) using mouse microarrays and comparing winter torpid with summer active arctic ground squirrels. Among them, the genes involved in non-shivering thermogenesis (NST) were significantly up-regulated, whereas those involved in protein synthesis were significantly down-regulated (Yan et al. 2006). However, mouse (Mus musculus) only share on average 89% mRNA sequence identities with arctic ground squirrel. Although the mouse microarray study generated a large number of candidate genes, heterologous hybridization may have produced both relatively high false positive and negative results.

### SUMMARY OF THE INVENTION

A need exists in the field to identify specific genes involved in the hibernation metabolic state, including those genes involved in respiratory, cardiovascular, immunological and neurological responses to metabolic suppression during hibernation. A further need exists to identify the protein products of such genes as well as inhibitors or activators of such genes and/or their expressed proteins. Identification of hibernation-related genes provides targets for therapeutic treatment of a wide variety of disease states and/or metabolic conditions, while identification of activators and/or inhibitors of such genes or their expressed proteins provides candidate pharmacologic agents of use in such therapies.

The present invention fulfills this unresolved need in the art by providing methods and compositions for identifying and/or detecting hibernation-related genes and their encoded proteins; inhibitors and activators thereof and probes, primers, antibodies and other ways to detect expression of hibernation-related genes. In various embodiments, inhibitors and/or activators of hibernation-related genes or their expressed protein products may be used for therapeutic treatment of a variety of diseases that are responsive to manipulation of hibernation-related metabolic pathways and/or regulatory mechanisms. In particular embodiments, the hibernation-related genes are selected from the group consisting of Adfp, Atf4, Cact, Myl6, Ca3, Ckm, Rps2, Lgmn, Fabpa, Fabph and Cyp51a1. In a more particular embodiment, the hibernation-related gene is Myl6. The Myl6 gene and its protein product may be of particular use for predication, diagnosis or therapy of diseases relating to cardiac function.

### BRIEF DESCRIPTION OF THE DRAWINGS

The following drawings form part of the present specification and are included to further demonstrate certain aspects of particular embodiments of the invention. The embodiments may be better understood by reference to one or more of these drawings in combination with the detailed description presented herein.

FIG. 1. (A) Torpor-arousal cycles in a hibernating arctic ground squirrel. (B) Enlargement showing the four stages in a torpor-arousal cycle during hibernation in a telemetrized animal.

FIG. 2. Four genes with significant modulation of expression during torpor-arousal cycle of hibernation. (A) Adfp in BAT, (B) Atf4 in liver, (C) Cact/Slc25a20 in heart, (D) Cyp51a1 in hypothalamus during early arousal (EA), late arousal (LA), early torpor (ET), late torpor (LT), and post-reproduction (P) as measured by both Illumina beadarrays and Real-time PCR (QPCR). All of four genes showed significant (P<0.05) modulation in four-stage analysis during torpor-arousal cycles in Real-time PCR (Table 5). The method to calculate the normalized gene expression values is given in the Materials and Methods section of Example 1.

Table 1. Sequence sources of the genes probed using the 1A and 2A arrays. 700 genes in addition to the seven house-keeping genes are represented on each array.

Table 2. Numbers of detected and differentially expressed genes in three tissues on Illumina 16-sample beadchips. Detection score>0.99 was used as the criterion for detection. T>P or T<P denotes that the gene expression in torpid animals is significantly (P<0.05) higher or lower than in post-reproductive animals in Welch two-sample t-test.

Table 3. Numbers of detected and differentially expressed genes in three-stage analysis in 96-sample Illumina array matrix. Detection score>0.99 was used as the criterion for detection. P value<0.05 in one-way ANOVA was used as the criterion for differential expression. A=Aroused animals; T=Torpid animals; P=Post-reproductive animals. X>Y or X<Y denotes that the gene expression in X is significantly (P<0.05) higher or lower than in Y in post hoc Tukey's test, where X, Y=(A, T, P).

Table 4. Numbers of differentially expressed genes tested in three-stage Real-time PCR assay. P value<0.05 in one-way ANOVA was used as the criterion for differential expression. A=Aroused animals; T=Torpid animals; P=Post-reproductive animals. X>Y or X<Y denotes that the gene expression in X is significantly (P<0.05) higher or lower than in Y in post hoc Tukey's test, where X, Y=(A, T, P).

Table 5. Differential gene expression patterns in three-stage analysis are represented by (x_{A-T}, x_{A-P}, x_{T-P}), where x_{I-J}=1 if the gene expression in stage I is significantly higher than that in stage J; −1 if significantly lower; 0 if not significantly different; I, J=A (aroused), T (torpid), P (post-reproductive). P<0.05 in post hoc Tukey's test is used as the criterion for significance. The superscripts on the gene symbols: B, L, HE, S, HY represent brown adipose tissue, liver, heart, skeletal muscle, and hypothalamus.

Table 6. Number of differentially expressed genes in four-stage analysis in Real-time PCR assay. P value<0.05 in one-way ANOVA was used as the criterion for differential expression. EA=Early Aroused animals; LA=Late Aroused animals; ET=Early Torpid animals; LT=Late torpid animals. X>Y or X<Y denotes that the gene expression in X is significantly (P<0.05) higher or lower than in Y in post hoc Tukey's test, where X, Y=(EA, LA, ET, LT).

### DESCRIPTION OF ILLUSTRATIVE EMBODIMENTS

All documents, or portions of documents, cited in this application, including but not limited to patents, patent applications, articles, books, and treatises, are hereby expressly incorporated by reference in their entirety. Additional details regarding the methods and compositions are disclosed in Yan et al. (2007, Physiol. Genom. 32:170-181), the entire contents of which are incorporated herein by reference.

### DEFINITIONS

As used herein, “a” or “an” may mean one or more than one of an item.

As used herein, the terms “and” and “or” may be used to mean either the conjunctive or disjunctive. That is, both terms should be understood as equivalent to “and/or” unless otherwise stated.

As used herein, “about” means any number within plus or minus ten percent of a value. For example, “about 100” would include any number between 90 and 110.

As used herein, a “hibernation-related gene” is a gene that is differentially expressed in hibernating versus active animals. Such differential expression may be determined by a wide variety of known techniques, such as differential display, subtraction hybridization, RT-PCR, Western blotting, quantitative binding to gene chips and other well-known methods. The skilled artisan will realize that differential expression may be determined by comparison of expression during different stages of the torpor-arousal cycle, such as early arousal, late arousal, early torpor, late torpor and post-reproduction. A “protein product” of a hibernation-related gene is any protein, peptide or polypeptide that is expressed from a hibernation-related gene. As used herein, “protein product” and “expressed protein” are used interchangeably and have the same meaning.

As used herein, an “activator” of a hibernation-related gene or protein product thereof encompasses any molecule, compound, composition, complex, or treatment that results in a net increase in the amount and/or activity of any protein product of a hibernation-related gene. An “inhibitor” of a hibernation-related gene or protein product thereof encompasses any molecule, compound, composition, complex, or treatment that results in a net decrease in the amount and/or activity of any protein product of a hibernation-related gene. The skilled artisan will realize that in some cases an activator or inhibitor may affect the levels of transcription, translation, post-translational processing, stability and/or degradation of the mRNA or protein products of a hibernation-related gene. In other cases an inhibitor or activator may interact directly or indirectly with one or more protein products of a hibernation-related gene, for example by affecting phosphorylation/dephosphorylation of proteins or by directly binding to a regulatory or active site on a protein. In still other cases, an inhibitor or activator may act by changing the relative levels of the various protein isoforms that are expressed from a particular hibernation-related gene. The skilled artisan will realize that different isoforms expressed from the same gene may exhibit different regulatory and/or catalytic properties or activities that affect cell metabolism. Such activators and inhibitors of hibernation-related genes or their protein products are of use in the claimed methods regardless of their mechanism of activation or inhibition.

### Hibernation-Related Genes

In most mammalian hibernators, hibernation is periodically interrupted by spontaneous arousal. However, the molecular mechanisms of hibernation and the function of arousal remain unclear. The Examples below describe a large-scale screening of hibernation-related differential gene expression in a wide range of tissues including brown adipose tissue, liver, heart, hypothalamus, and skeletal muscle in hibernating arctic ground squirrels. The screening compared four stages in torpor-arousal cycles and non-hibernating animals using both oligonucleotide array technology and real-time PCR assays.

Comparing torpid and aroused animals with non-hibernating animals, significant seasonal differences in gene expression were detected in the genes involved in glycolysis, fatty acid metabolism, gluconeogenesis, amino-acid metabolism, transport, detoxication, cardiac contractility, circadian rhythms, muscle dystrophy and RNA protection, among others, in various tissues. The results are in contrast to the hypothesis that mammalian hibernators arouse to replenish mRNA levels. Instead it was observed for the first time that complex modulation of gene expression occurs during multiple stages of torpor-arousal cycles.

In particular, most significant differences in gene expression during torpor-arousal cycles were observed during the transition from late torpor to early arousal. During this transition, the mRNA levels of a group of metabolic genes drops significantly, perhaps due to the exhaustion of mRNA transcripts during the energetic demands of the early arousal phase. In contrast, the mRNA levels for the genes related to cell growth and proliferation rises sharply during this transition, which may reflect the resumption of the cell cycle process during arousal after it has been stalled during torpor.

The skilled artisan will realize that the differentially expressed hibernation-related genes and/or their protein products that are identified using the methods disclosed herein provide targets for therapeutic intervention in a wide range of diseases or conditions, such as cardiac, respiratory, neurologic, metabolic and/or immunologic diseases or conditions. Inhibitors or activators of such genes and proteins may be identified using a variety of techniques known in the art. The hibernation-related genes identified in the Examples below are not meant to be exhaustive and other such genes may be identified using the disclosed methods, within the scope of the claimed subject matter. However, in certain embodiments the hibernation-related genes may be selected from the group consisting of Adfp, Atf4, Cact, Myl6, Ca3, Ckm, Rps2, Lgmn, Fabpa, Fabph and Cyp51a1. In more particular embodiments, the hibernation-related gene may be Myl6.

Inhibitors and Activators of Hibernation-Related Genes and/or Their Protein Products

Various embodiments concern inhibitors or activators of hibernation-related genes or their expressed proteins that are useful for the treatment of human diseases and pathological conditions. Agents that inhibit or activate hibernation-related genes or their expressed proteins may be used in combination with other therapeutic agents to enhance their therapeutic effects or decrease potential side effects. In certain embodiments, compounds that have been reported to affect the activities of hibernation-related genes, such as thyronamine analogs or derivatives, bradykinin, or fibrinopeptide A (FPA) may be utilized (see U.S. Pat. No. 6,979,750, and U.S. Patent Application Publication No. 20030228371, each incorporated herein by reference). However, the instant methods may also comprise the discovery and use of novel inhibitors or activators of hibernation-related genes or their protein products.

In one aspect, the present invention provides compositions and methods useful for treating diseases and conditions related to the activities of hibernation-related genes or their expressed proteins. These diseases may include, but are not limited to, ischemia, reperfusion injury, congestive heart failure, cardiomyopathy, myocardial infarction, atherosclerosis, cardiac arrhythmia, tachycardia, bradycardia, hypertension, hyperthermia, hypothermia, fever, heatstroke, menopausal hot flashes, hyperthyroidism, hypothyroidism, retinopathy, macular degeneration, glaucoma, stroke, obesity, diabetes, osteoporosis, lipidemias, hyperlipidemia, hypercholesterolemia, weight loss, esophageal reflux disease, diarrhea, other diseases involving gastrointestinal motility, cancer, leukemia, lymphoma, acute lymphocytic leukemia, acute myelogenous leukemia, chronic lymphocytic leukemia, chronic myelogenous leukemia, anemia, shock, hypovolemic shock, rheumatoid arthritis, chronic inflammatory disorders, asthma, muscular dystrophy, Duchenne muscular dystrophy, renal failure, cirrhosis of the liver, infertility, erectile dysfunction, neurologic disease, bipolar disease, depression, schizophrenia, eating disorders, bulemia, anxiety, seizure, epilepsy, insomnia, other sleeping disorders, migraine headache, attention deficit disorder, Parkinsonism and Alzheimer's disease.

In some embodiments, inhibitors or activators of the identified hibernation-related genes may be known in the art and any such known inhibitors or activators may be used in the practice of the claimed methods. For example, the activity of the Adfp gene and/or protein products has been reported to be regulated by long-chain polyunsaturated fatty acids (Tobin et al., 2006, J. Lipid Res. 47:815-23); VLDL, triacsin C, triacylglycerol or oleic acid (Masuda et al., 2006, J. Lipid Res. 47:87-98); PPARα-agonists (Dalen et al., 2006, J. Lipid Res. 47:931-943); and PPARγ-ligands such as troglitazone as well as LG268 (Bildirici et al., 2003, J. Clin. Metab. 88:6056-62). The activity of the Atf4 gene and/or protein products has been reported to be regulated by arsenite (Roybal et al., 2005, J. Biol. Chem. 280:20331-339); Mitocin/CENP-F (Zhou et al., 2005, J. Biol. Chem. 280:13973-977); and cisplatin (Tanabe et al., 2003, Cancer Res. 63:8592-95). The activity of the Cyp51a1 gene and/or protein products has been reported to be regulated by azole anti-fungal agents such as ketoconazole and fluconazole (Matsuura et al., 2005, J. Biol. Chem. 280:9088-96) and oxysterols (Stromstedt et al., Arch. Biochem. Biophys. 1996, 329:73-81. The activity of the Ca3 gene and/or protein products has been reported by be regulated by aminobenzolamide (Vidgren et al., Int. J. Biol. Macromol. 1993, 15:97-100).

Putative inhibitors or activators may be initially tested for binding activity associated with therapeutic or diagnostic use in vitro. For example, compositions may be tested for binding activity to protein products of hibernation-related genes by ELISA, flow cytometric assay, affinity column chromatography, solid-phase binding assay or any other binding assays known in the art. The ability of putative inhibitors or activators to affect expression of hibernation-related genes may be determined by known assays, as described in more detail below. For example, model cell lines or intact organs or tissues may be assayed for the levels of expressed proteins in the presence or absence of putative inhibitors or activators using antibodies against one or more protein products of hibernation-related genes. Where such protein products have known catalytic or regulatory activities, the effects of putative inhibitors or activators on such activities may be determined using well known techniques, such as enzyme activity assays.

Assays to Screen for Inhibitors or Activators

For convenience, a putative inhibitor or activator may be referred to below as a test molecule(s). Several types of in vitro assays may be performed using the purified or semi-purified protein products of hibernation-related genes. In one such assay, purified protein or a fragment thereof may be immobilized by attachment to the bottom of the wells of a microtiter plate. The test molecule(s) can then be added either one at a time or simultaneously to the wells. After incubation, the wells can be washed and assayed to determine the degree of protein binding to the test molecule. Binding may be determined by a multiplicity of known techniques, for example by “tagging” the test molecule(s) with a detectable radioactive, fluorescent, luminescent or other label. In variations of such assays, the test molecule(s) may be attached to the solid substrate and purified or semi-purified protein product added. Binding of protein to the substrate may be monitored, for example, using labeled primary or secondary antibodies against the protein of interest. Typically, the molecule will be tested over a range of concentrations, and a series of control wells lacking one or more elements of the test assays are used to detect non-specific binding.

In certain embodiments, the expressed hibernation-related protein may bind to one or more other cellular proteins, for example in a ligand-receptor type of interaction. The activator or inhibitor may act by interfering with or facilitating the ligand-receptor binding interaction. In such cases, an alternative to microtiter plate type of binding assays comprises immobilizing hibernation-related expressed proteins (or their receptors) on agarose beads, acrylic beads or other types of such inert substrates. The inert substrate to which the protein is attached may be placed in a solution containing the test molecule and the complementary pair of the ligand-receptor complex. After incubation, the inert substrate can be collected by centrifugation, and the amount of binding of ligand to receptor can be readily assessed. Alternatively, the inert substrate complex can be immobilized in a column and the test molecule and expressed protein (or receptor) passed over the column. Formation of the ligand-receptor complex can then be assessed using any known techniques, i.e., radiolabeling, antibody binding, or the like. In another alternative, the ligand-receptor complex may be attached via one member of the pair to an inert substrate and the ability of the test molecule to displace the bound ligand or receptor assayed.

Another type of in vitro assay that is useful for identifying molecules that inhibit ligand-receptor binding activity is the Biacore Assay System (Pharmacia, Piscataway, N.J.), which uses a surface plasmon resonance detector system. This assay essentially involves covalent binding of either hibernation-related expressed protein or receptor protein to a dextran-coated sensor chip which is located in the detector. The test molecule and the complementary component can then be injected into the chamber containing the sensor chip either simultaneously or sequentially, and the amount of binding of hibernation-related expressed protein/receptor protein can be assessed based on the change in molecular mass which is physically associated with the dextran-coated side of the of the sensor chip. The change in mass is detectable by a corresponding change in surface plasmon resonance.

According to certain embodiments, one may expose a cell line that expresses hibernation-related proteins to test molecules to determine whether the production of expressed proteins in the cell line is reduced or increased, and/or some metabolic or other activity of the cell line is affected. The levels or activities of expressed proteins may be compared in treated and untreated cell lines either by assaying for the amount of expressed protein produced (e.g., by Western blotting or other known technique) or by assaying for a known activity of the expressed protein. Effects on gene transcription may also be readily determined using techniques such as reverse transcriptase-polymerase chain reaction techniques, RNAse protection assays and the like.

Binding Assays

Binding assays are of use for a variety of purposes, such as assaying the ability of putative inhibitors or activators to bind to the protein product(s) of a hibernation-related gene. Alternatively, binding assays may be utilized for diagnostic purposes, for example to quantify the amount of protein product(s) of a given hibernation-related gene expressed in a particular cell, organ or tissue, where the level of expression is indicative of the presence or absence of a particular disease. Immunological binding assays typically utilize a capture agent to bind specifically to and often immobilize the target antigen. In one embodiment, the capture agent is an antibody or antigen-binding region thereof that specifically binds to a hibernation-related expressed protein. Methods and compositions to perform immunological binding assays are well known in the art [e.g., Asai, ed., Methods in Cell Biology, Vol. 37, Antibodies in Cell Biology, Academic Press, Inc., New York (1993); Harlowe and Lane, 1988, Antibodies: A Laboratory Manual, Cold Spring Harbor Laboratory]. Although the present section generally focuses on antibodies or antibody fragments as the capture agent, the skilled artisan will realize that other types of specific or selective binding moieties, such as aptamers or affibodies, are known and may be similarly utilized. Such alternative capture agents are described in more detail below.

Immunological binding assays frequently utilize a labeling agent that will signal the existence of the bound complex formed by the capture agent and antigen. The labeling agent can be one of the molecules comprising the bound complex; i.e. it can be a labeled specific binding antibody. Alternatively, the labeling agent can be a third molecule, commonly a labeled second antibody, which binds to the bound complex. For example, the second antibody can be modified with a detectable moiety, such as biotin, which can then be bound by a fourth molecule, such as enzyme-labeled streptavidin. Other proteins capable of specifically binding immunoglobulin constant regions, such as protein A or protein G may also be used as the labeling agent. These binding proteins exhibit a strong non-immunogenic reactivity with immunoglobulin constant regions from a variety of species. Akerstrom, J. Immunol. 135:2589-2542 (1985); Chaubert, Mod. Pathol. 10:585-591 (1997).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays will be carried out at ambient temperature, although they can be conducted over a range of temperatures.

Non-Competitive Binding Assays

Immunological binding assays can be of the non-competitive type. These assays have an amount of captured analyte that is directly measured. For example, in one preferred “sandwich” assay, the capture agent (antibody) can be bound directly to a solid substrate where it is immobilized. These immobilized capture agents then capture (bind to) antigen present in the test sample. The protein thus immobilized is then bound to a labeling agent, such as a second antibody having a label. In another preferred “sandwich” assay, the second antibody lacks a label, but can be bound by a labeled antibody specific for antibodies of the species from which the second antibody is derived. The second antibody also can be modified with a detectable moiety, such as biotin, to which a third labeled molecule can specifically bind, such as streptavidin. [See Harlow and Lane, Antibodies, A Laboratory Manual, Ch 14, Cold Spring Harbor Laboratory, NY (1988), incorporated herein by reference.]

Competitive Binding Assays

Immunological binding assays can be of the competitive type. The amount of analyte present in the sample is measured indirectly by measuring the amount of an added analyte displaced, or competed away, from a capture agent (e.g., antibody) by the analyte present in the sample. In one preferred competitive binding assay, a known amount of analyte, usually labeled, is added to the sample and the sample is then contacted with the capture agent. The amount of labeled analyte bound to the antibody is inversely proportional to the concentration of analyte present in the sample. [See, Harlow and Lane, 1988, Ch 14, pp. 579-583.]

In another preferred competitive binding assay, the capture agent is immobilized on a solid substrate. The amount of protein bound to the capture agent may be determined either by measuring the amount of protein present in a protein/antibody complex, or alternatively by measuring the amount of remaining uncomplexed protein. The amount of protein may be detected by providing a labeled protein (Id.)

Yet another preferred competitive binding assay, hapten inhibition is utilized. Here, a known analyte is immobilized on a solid substrate. A known amount of antibody is added to the sample, and the sample is contacted with the immobilized analyte. The amount of antibody bound to the immobilized analyte is inversely proportional to the amount of analyte present in the sample. The amount of immobilized antibody may be detected by detecting either the immobilized fraction of antibody or the fraction that remains in solution. Detection may be direct where the antibody is labeled or indirect by the subsequent addition of a labeled moiety that specifically binds to the antibody as described above.

### Aptamers

In certain embodiments, an inhibitor, activator or binding agent of use may be an aptamer. Aptamers are usually single-stranded, short molecules of RNA, DNA or a nucleic acid analog, that may adopt three-dimensional conformations complementary to a wide variety of target molecules. Methods of constructing and determining the binding characteristics of aptamers are well known in the art. For example, such techniques are described in U.S. Pat. Nos. 5,582,981, 5,595,877 and 5,637,459, each incorporated herein by reference.

Aptamers may be prepared by any known method, including synthetic, recombinant, and purification methods, and may be used alone or in combination with other ligands specific for the same target. In general, a minimum of approximately 3 nucleotides, preferably at least 5 nucleotides, are necessary to effect specific binding. Aptamers of sequences shorter than 10 bases may be feasible, although aptamers of 10, 20, 30 or 40 nucleotides may be preferred.

Aptamers need to contain the sequence that confers binding specificity, but may be extended with flanking regions and otherwise derivatized. In preferred embodiments, the target-binding sequences of aptamers may be flanked by primer-binding sequences, facilitating the amplification of the aptamers by PCR or other amplification techniques. In a further embodiment, the flanking sequence may comprise a specific sequence that preferentially recognizes or binds a moiety to enhance the immobilization of the aptamer to a substrate. Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR₂, P(O)R, P(O)OR′, CO, or CNR₂, wherein R is H or alkyl (1-20C) and R′ is alkyl (1-20C); in addition, this group may be attached to adjacent nucleotides through O or S. Not all linkages in an oligomer need to be identical.

The aptamers used as starting materials in the process to determine specific binding sequences may be single-stranded or double-stranded DNA or RNA. In a preferred embodiment, the sequences are single-stranded DNA, which is less susceptible to nuclease degradation than RNA. In preferred embodiments, the starting aptamer will contain a randomized sequence portion, generally including from about 10 to 400 nucleotides, more preferably 20 to 100 nucleotides. The randomized sequence is flanked by primer sequences that permit the amplification of aptamers found to bind to the target. For synthesis of the randomized regions, mixtures of nucleotides at the positions where randomization is desired may be added during synthesis.

Methods for preparation and screening of aptamers that bind to particular targets of interest are well known, for example U.S. Pat. No. 5,475,096 and U.S. Pat. No. 5,270,163, each incorporated by reference. The technique generally involves selection from a mixture of candidate aptamers and step-wise iterations of binding, separation of bound from unbound aptamers and amplification. Because only a small number of sequences (possibly only one molecule of aptamer) corresponding to the highest affinity aptamers exist in the mixture, it is generally desirable to set the partitioning criteria so that a significant amount of aptamers in the mixture (approximately 5-50%) are retained during separation. Each cycle results in an enrichment of aptamers with high affinity for the target. Repetition for between three to six selection and amplification cycles may be used to generate aptamers that bind with high affinity and specificity to the target. Aptamers may be selected to bind to and inhibit or activate one or more proteins products of hibernation-related genes.

### Phage Display

Alternatively, short peptide sequences that bind to hibernation-related protein products may be prepared by the phage display technique. Such short binding peptides may also be of use as inhibitors, activators or binding agents. Various methods of phage display and techniques for producing diverse populations of binding peptides are well known in the art. For example, U.S. Pat. Nos. 5,223,409; 5,622,699 and 6,068,829, each of which is incorporated herein by reference, disclose methods for preparing a phage library. The phage display technique involves genetically manipulating bacteriophage so that small peptides can be expressed on their surface (Smith and Scott, 1985, Science 228:1315-1317; Smith and Scott, 1993, Meth. Enzymol. 21:228-257).

The past decade has seen considerable progress in the construction of phage-displayed peptide libraries and in the development of screening methods in which the libraries are used to isolate peptide ligands. For example, the use of peptide libraries has made it possible to characterize interacting sites and receptor-ligand binding motifs within many proteins, such as antibodies involved in inflammatory reactions or integrins that mediate cellular adherence. This method has also been used to identify novel peptide ligands that may serve as leads to the development of peptidomimetic drugs or imaging agents (Arap et al., 1998, Science 279:377-380). In addition to peptides, larger protein domains such as single-chain antibodies may also be displayed on the surface of phage particles (Arap et al., 1998).

Targeting amino acid sequences selective for a given target molecule may be isolated by panning (Pasqualini and Ruoslahti, 1996, Nature 380:364-366; Pasqualini, 1999, The Quart. J. Nucl. Med. 43:159-162). In brief, a library of phage containing putative targeting peptides is administered to isolated cell types or target molecules and samples containing bound phage are collected. Phage that bind to a target may be eluted from a target cell type or target molecule and then amplified by growing them in host bacteria.

In certain embodiments, the phage may be propagated in host bacteria between rounds of panning. Rather than being lysed by the phage, the bacteria may instead secrete multiple copies of phage that display a particular insert. If desired, the amplified phage may be exposed to the target cell types or target molecule again and collected for additional rounds of panning. Multiple rounds of panning may be performed until a population of selective or specific binders is obtained. The amino acid sequence of the peptides may be determined by sequencing the DNA corresponding to the targeting peptide insert in the phage genome. The identified targeting peptide may then be produced as a synthetic peptide by standard protein chemistry techniques (Arap et al., 1998, Smith et al., 1985).

In some embodiments, a subtraction protocol may be used to further reduce background phage binding. The purpose of subtraction is to remove phage from the library that bind to targets other than the target of interest. In alternative embodiments, the phage library may be prescreened against a control cell, tissue or organ. After subtraction the library may be screened against the molecule or cell of interest. Other methods of subtraction protocols are known and may be used in the practice of the claimed methods, for example as disclosed in U.S. Pat. Nos. 5,840,841, 5,705,610, 5,670,312 and 5,492,807, incorporated herein by reference.

### Regulation of Endogenous Gene Expression

In certain embodiments, an inhibitor of a hibernation-related gene may act by inhibiting transcription of the gene, for example using anti-sense technology or small inhibitory RNA (siRNA). As is well known, nucleic acid may be expressed in either sense or anti-sense orientation. Antisense technology may be conveniently used to inhibit gene expression. To accomplish this, a nucleic acid segment from the desired gene is cloned and operably linked to a promoter such that the anti-sense strand of RNA will be transcribed. The construct is then transformed into target cells and the antisense strand of RNA is produced. Antisense RNA inhibits gene expression by preventing the accumulation of mRNA which encodes a polypeptide of interest. [See, e.g., Sheehy et al., Proc. Natl. Acad. Sci. (USA) 85: 8805-8809 (1988); Hiatt et al., U.S. Pat. No. 4,801,340.]

Another method of suppression is sense suppression. Introduction of nucleic acid configured in the sense orientation has been shown in some cases to block the transcription of target genes. [See, e.g., U.S. Pat. No. 5,034,323.]

Catalytic RNA molecules or ribozymes may also be used to inhibit expression of genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is disclosed, for example, in Haseloff et al., Nature 334:585-591 (1988).

Co-Suppression

In certain embodiments, nucleotide sequences of use may be provided in transcriptional units as co-suppression cassettes for transcription in a cell of interest. Transcription units may contain coding and/or non-coding regions of the genes of interest. Additionally, transcription units may contain promoter sequences with or without coding or non-coding regions. The co-suppression cassette may include 5′ (but not necessarily 3′) regulatory sequences, operably linked to at least one nucleotide sequence to be transcribed. Co-suppression cassettes of use may comprise sequences in so-called “inverted repeat” structures. The cassette may additionally contain a second copy of the fragment in opposite direction to form an inverted repeat structure. Opposing arms of the structure may or may not be interrupted by any nucleotide sequence related or unrelated to the nucleotide sequences of the target (see Fiers et al. U.S. Pat. No. 6,506,559). The transcriptional units are linked to be co-transformed into the organism. Alternatively, additional transcriptional units may be provided on multiple over-expression and/or co-suppression cassettes.

The technique of transgenic co-suppression may be used to reduce or eliminate the level of at least one expressed protein. One method of transgenic co-suppression comprises transforming a cell with at least one transcriptional unit containing an expression cassette with a promoter that drives transcription, operably linked to at least one nucleotide sequence transcript in the sense orientation that encodes at least a portion of the protein of interest. Methods for suppressing gene expression using nucleotide sequences in the sense orientation are known in the art. The methods generally involve transforming cells with a DNA construct comprising a promoter that drives transcription, operably linked to at least a portion of a nucleotide sequence that corresponds to the transcript of the endogenous gene. Typically, such a nucleotide sequence has substantial sequence identity to the sequence of the transcript of the endogenous gene, at least about 65%, 70%, 75%, 80%, 85%, 90%, 91%, 92%, 93%, 94%, 95%, 96%, 97%, 98%, 99% or 99.5% or more sequence identity over the entire length of the sequence. Furthermore, portions, rather than the entire nucleotide sequence, of the polynucleotides may be used to disrupt the expression of the target gene product. Generally, sequences of at least 10, 15, 20, 21, 22, 23, 24, 25, 26, 27, 28, 29, 30, 35, 40, 45, 50, 55, 60, 65, 70, 75, 80, 85, 90, 95, 100, 200 nucleotides, or greater may be used. [See U.S. Pat. Nos. 5,283,184 and 5,034,323; herein incorporated by reference.]

Additional techniques of co-suppression are known in the art and may be similarly applied in the claimed methods. These techniques involve the silencing of a targeted gene by spliced hairpin RNA's and similar methods, also called RNA interference or promoter silencing (see Smith et al. (2000) Nature 407:319-320; Waterhouse and Helliwell (2003) Nat. Rev. Genet. 4:29-38; Waterhouse et al. (1998) Proc. Natl. Acad. Sci. USA 95:13959-13964; Chuang and Meyerowitz (2000) Proc. Natl. Acad. Sci. USA 97:4985-4990; Stoutjesdijk et al. (2002) Physiol. 129:1723-1731; and Patent Application WO 99/53050; WO 99/49029; WO 99/61631; WO 00/49035 and U.S. Pat. No. 6,506,559, each of which is incorporated herein by reference).

The expression cassette for co-suppression may be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, International Publication No. WO 02/00904, herein incorporated by reference.

RNAi

In other embodiments, inhibition of the expression of a protein of interest may be obtained by RNA interference, for example by expression of a gene encoding a small inhibitory RNA (siRNA). siRNAs are regulatory agents consisting of about 22 ribonucleotides. siRNA is often highly efficient at inhibiting the expression of endogenous genes. [See, for example Javier et al. (2003) Nature 425: 257-263, incorporated herein by reference.]

In another embodiment, the polynucleotide to be introduced into the target cell may comprise an inhibitory sequence that encodes a zinc finger protein that binds to a gene encoding a protein, resulting in reduced expression of the gene. In particular embodiments, the zinc finger protein binds to a regulatory region of a hibernation-related gene. In other embodiments, the zinc finger protein binds to a messenger RNA encoding a protein and prevents its translation. Methods of selecting sites for targeting by zinc finger proteins have been disclosed, for example, in U.S. Pat. No. 6,453,242, and methods for using zinc finger proteins to inhibit the expression of genes are disclosed, for example, in U.S. Patent Publication No. 20030037355.

Methods for antisense suppression may be used to reduce or eliminate the level of at least one protein. The methods of antisense suppression comprise transforming a cell with at least one expression cassette comprising a promoter that drives expression in the cell, operably linked to at least one nucleotide sequence that is antisense to a nucleotide sequence transcript of a target gene. By “antisense suppression” is intended the use of nucleotide sequences that are antisense to nucleotide sequence transcripts of endogenous genes to suppress the expression of those genes. Methods for suppressing gene expression using nucleotide sequences in the antisense orientation are known in the art and any such known method may be used.

In certain embodiments, RNAi inhibitors for identified hibernation-related genes may be commercially available and any such commercial compounds may be used in the practice of the claimed methods. For example, RNAi inhibitors targeted to the Adfp, Atf4, CACT, Myl6, Ca3, Ckm and Cyp51a1 genes may be purchased from Invitrogen (Carlsbad, Calif.).

### Vectors for Cloning, Gene Transfer and Expression

In certain embodiments, expression vectors may be employed to express peptides, proteins or RNAs. For example, an inhibitory or activating binding peptide may be inserted into an expression vector and transformed into target cells. In other embodiments, the expression vectors may be used, for example, in gene therapy by expressing one or more protein products of a hibernation-related gene in a target cell, or alternatively by expressing an siRNA or other inhibitory RNA molecule. Expression requires that appropriate signals be provided in the vectors, which include various regulatory elements, such as enhancers/promoters from either viral or mammalian sources that drive expression of the genes of interest in host cells. Elements designed to optimize messenger RNA stability and/or translatability in host cells also are known and may be used.

### Regulatory Elements

The terms “expression construct” or “expression vector” are meant to include any type of genetic construct containing a nucleic acid coding for a gene product in which part or all of the nucleic acid coding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent and under the control of a promoter that is capable of being expressed in a human cell. Generally speaking, such a promoter might include either a human or viral promoter. In various embodiments, the human cytomegalovirus (CMV) immediate early gene promoter, the SV40 early promoter, the Rous sarcoma virus long terminal repeat, rat insulin promoter, and glyceraldehyde-3-phosphate dehydrogenase promoter can be used to obtain high-level expression of the coding sequence of interest. The use of other viral or mammalian cellular or phage promoters which are well-known in the art to achieve expression of a coding sequence of interest is contemplated as well, provided that the levels of expression are sufficient for a given purpose.

Where a cDNA insert is employed, typically one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

Selectable Markers

In certain embodiments, the cells containing nucleic acid constructs may be identified in vitro or in vivo by including a marker in the expression construct. Such markers would confer an identifiable change to the transformed cell, permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin, and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

Delivery of Expression Vectors

There are a number of ways in which expression vectors may be introduced into cells. In certain embodiments, the expression construct comprises a virus or engineered construct derived from a viral genome. The ability of certain viruses to enter cells via receptor-mediated endocytosis, to integrate into the host cell genome, and to express viral genes stably and efficiently have made them attractive candidates for the transfer of foreign genes into mammalian cells (Ridgeway, In: Vectors: A Survey of Molecular Cloning Vectors and Their Uses, Rodriguez et al., eds., Stoneham: Butterworth, pp. 467-492, 1988; Nicolas and Rubenstein, In: Vectors: A survey of molecular cloning vectors and their uses, Rodriguez and Denhardt, eds., Stoneham: Butterworth, pp. 494-513, 1988; Baichwal and Sugden, 1986, In: Gene Transfer, Kucherlapati R, ed., New York, Plenum Press, pp. 117-148; Temin, In: Gene Transfer, Kucherlapati R, ed., New York, Plenum Press, pp. 149-188, 1986). Preferred gene therapy vectors are generally viral vectors, such as adenoviral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host cell replication for gene expression, making them ideally suited for rapid, efficient, heterologous gene expression. Techniques for preparing replication-deficient infective viruses are well known in the art.

In using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

DNA viruses used as gene vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986). One of the preferred methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

Generation and propagation of adenovirus vectors which are replication deficient depend on a helper cell line, designated 293, which is transformed from human embryonic kidney cells by Ad5 DNA fragments and constitutively expresses E1 proteins (Graham et al., J. Gen. Virol., 36:59-72, 1977). Since the E3 region is dispensable from the adenovirus genome (Jones and Shenk, Cell, 13:181-188, 1978), adenovirus vectors, with the help of 293 cells, carry foreign DNA in either the E1, the E3, or both regions (Graham and Prevec, In: Methods in Molecular Biology: Gene Transfer and Expression Protocol, E. J. Murray, ed., Humana Press, Clifton, N.J., 7:109-128, 1991).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As discussed, a preferred helper cell line is 293.

Other gene transfer vectors may be constructed from retroviruses. The retroviruses are a group of single-stranded RNA viruses characterized by an ability to convert their RNA to double-stranded DNA in infected cells by a process of reverse-transcription (Coffin, In: Virology, Fields et al., eds., Raven Press, New York, pp. 1437-1500, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences, and also are required for integration in the host cell genome (Coffin, 1990).

In order to construct a retroviral vector, a nucleic acid encoding a protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., Cell, 33:153-159, 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., Virology, 67:242-248, 1975).

Other viral vectors may be employed as expression constructs. Vectors derived from viruses such as vaccinia virus (Ridgeway, 1988; Baichwal and Sugden, 1986), adeno-associated virus (AAV) (Ridgeway, 1988; Baichwal and Sugden, 1986; Hermonat and Muzycska, Proc. Natl. Acad. Sci. USA, 81:6466-6470, 1984), and herpes viruses may be employed. They offer several attractive features for various mammalian cells (Friedmann, Science, 244:1275-1281, 1989; Ridgeway, 1988; Baichwal and Sugden, 1986; Horwich et al., J. Virol., 64:642-650, 1990).

Alternative techniques for transformation of eukaryotic cells are known and may be used, including but not limited to electroporation, particle gun transformation, protoplast transformation, PEG mediated transformation and other well known methods.

### Methods of Disease Tissue Detection, Diagnosis and Imaging

Protein Based in Vitro Diagnosis

In certain embodiments, binding agents specific for hibernation-related protein products may be used to screen biological samples in vitro and/or in vivo for the presence of the expressed protein(s). In exemplary immunoassays, the target hibernation-related protein product or an antibody, fusion protein, or fragment specific for the protein product may be utilized in liquid phase or bound to a solid-phase carrier, as described below. In preferred embodiments, particularly those involving in vivo administration, the antibody or fragment thereof is humanized. Still more preferred, the fusion protein comprises a humanized or fully human antibody. The skilled artisan will realize that a wide variety of techniques are known for determining levels of expression of a particular gene and any such known method, such as immunoassay, RT-PCR, mRNA purification and/or cDNA preparation followed by hybridization to a gene expression assay chip may be utilized to determine levels of expression in individual subjects and/or tissues.

One example of a screening method for determining whether a biological sample contains an antigen of interest is radioimmunoassay (RIA). For example, in one form of RIA, the substance under test is mixed with antibody in the presence of radiolabeled antigen. In this method, the concentration of the test substance will be inversely proportional to the amount of labeled antigen bound to the antibody and directly related to the amount of free, labeled antigen. Other suitable screening methods will be readily apparent to those of skill in the art.

Alternatively, in vitro assays may be performed in which a ligand, antibody, fusion protein, or fragment thereof is bound to a solid-phase carrier. For example, antibodies can be attached to a polymer, such as aminodextran, in order to link the antibody to an insoluble support such as a polymer-coated bead, a plate or a tube.

The presence of the antigen in a biological sample may be determined using an enzyme-linked immunosorbent assay (ELISA). In the direct competitive ELISA, a pure or semipure antigen preparation is bound to a solid support that is insoluble in the fluid or cellular extract being tested and a quantity of detectably labeled soluble antibody, antibody fragment or ligand is added to permit detection and/or quantitation of the binary complex formed between solid-phase antigen and labeled binding molecule.

A sandwich ELISA requires small amounts of antigen, and the assay does not require extensive purification of the antigen. Thus, the sandwich ELISA is preferred to the direct competitive ELISA for the detection of an antigen in a clinical sample. See, for example, Field et al., Oncogene 4:1463 (1989); Spandidos et al., AntiCancer Res. 9: 821 (1989).

In a sandwich ELISA, a quantity of unlabeled antibody or antibody fragment (the “capture antibody”) is bound to a solid support, the test sample is brought into contact with the capture antibody, and a quantity of detectably labeled soluble antibody (or antibody fragment) is added to permit detection and/or quantitation of the ternary complex formed between the capture antibody, antigen, and labeled antibody. An antibody fragment is a portion of an antibody such as F(ab′)₂, F(ab)₂, Fab′, Fab, and the like. In the present context, an antibody fragment is a portion of an antibody that binds to an epitope of the antigen. The term “antibody fragment” also includes any synthetic or genetically engineered protein that acts like an antibody by binding to a specific antigen to form a complex. For example, antibody fragments include isolated fragments consisting of the light chain variable region, “Fv” fragments consisting of the variable regions of the heavy and light chains, and recombinant single chain polypeptide molecules in which light and heavy variable regions are connected by a peptide linker. An antibody fusion protein is a recombinantly produced antigen-binding molecule in which two or more of the same or different single-chain antibody or antibody fragment segments with the same or different specificities are linked. The fusion protein may comprise a single antibody component, a multivalent or multispecific combination of different antibody components or multiple copies of the same antibody component. The fusion protein may additionally comprise an antibody or an antibody fragment conjugated to a diagnostic/detection and/or a therapeutic agent. The term antibody includes humanized, human, chimeric and murine antibodies, antibody fragments thereof, immunoconjugates and fragments thereof and antibody fusion proteins and fragments thereof. Methods of performing a sandwich ELISA are well-known. See, for example, Field et al., supra, Spandidos et al., supra, and Moore et al., “Twin-Site ELISAs for fos and myc Oncoproteins Using the AMPAK System,” in METHODS IN MOLECULAR BIOLOGY, VOL. 10, pages 273-281 (The Humana Press, Inc. 1992). The skilled artisan will realize that an assay similar to a sandwich ELISA may be performed by substituting ligand for either the first unlabeled antibody or the second labeled antibody.

In other embodiments, Western blot analysis may be used to detect and quantify the presence of target antigens in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies or ligands that specifically bind the selected target. These antibodies or ligands may be directly labeled or alternatively may be subsequently detected using labeled secondary antibodies that specifically bind to the primary antibody or ligand.

In situ detection with histological samples can be used to determine the presence of the antigen and to determine the distribution of the antigen in the examined tissue. In situ detection can be accomplished by applying a detectably-labeled ligand or antibody to frozen or paraffin-embedded tissue sections. General techniques of in situ detection are well-known to those of ordinary skill. See, for example, Ponder, “Cell Marking Techniques and Their Application,” in MAMMALIAN DEVELOPMENT: A PRACTICAL APPROACH 113-38 Monk (ed.) (IRL Press 1987).

The ligands, antibodies, fusion proteins, and fragments thereof can be detectably labeled with any appropriate marker moiety, for example, a radioisotope, an enzyme, a fluorescent label, a dye, a chromagen, a chemiluminescent label, a bioluminescent label or a paramagnetic label. Methods of making and detecting such detectably-labeled antibodies are well-known to those of ordinary skill in the art, and are described in more detail below. The binding of marker moieties to antibodies can be accomplished using standard techniques known to the art. Typical methodology in this regard is described by Kennedy et al., Clin. Chim. Acta 70:1 (1976), Schurs et al., Clin. Chim. Acta 81: 1 (1977), Shih et al., Int'l J. Cancer 46: 1101 (1990).

Nucleic Acid Based in Vitro Diagnosis

In particular embodiments, nucleic acids may be analyzed to determine levels of expression, particularly using nucleic acid amplification methods. Nucleic acid sequences (mRNA and/or cDNA) to be used as a template for amplification may be isolated from cells contained in a biological sample, according to standard methodologies. The nucleic acid may be fractionated or whole cell RNA. Where RNA is used, it may be desired to convert the RNA to a complementary cDNA. In one embodiment, the RNA is whole cell RNA and is used directly as the template for amplification.

In one example, the determination of expression is performed by amplifying (e.g. by PCR) the mRNA or cDNA sequences and detecting and/or quantifying an amplification product by any methods known in the art, including but not limited to TaqMan assay (Applied Biosystems, Foster City, Calif.), agarose or polyacrylamide gel electrophoresis and ethidium bromide staining, hybridization to a microarray comprising a specific probe, Northern blotting, dot-blotting, slot-blotting, etc.

Various forms of amplification are well known in the art and any such known method may be used. Generally, amplification involves the use of one or more primers that hybridize selectively or specifically to a target nucleic acid sequence to be amplified. One of the best-known amplification methods is the polymerase chain reaction (referred to as PCR) which is described in detail in U.S. Pat. Nos. 4,683,195, 4,683,202 and 4,800,159.

One embodiment of the invention may comprise obtaining a suitable sample from an individual and detecting a messenger RNA. Once the tissue sample is obtained the sample may be prepared for isolation of the nucleic acids by standard techniques (e.g., cell isolation, digestion of membranes, Oligo dT isolation of mRNA etc.) The isolation of the mRNA may also be performed using kits known to the art (Pierce, AP Biotech, etc). A reverse transcriptase PCR amplification procedure may be performed in order to quantify an amount of mRNA amplified. Methods of reverse transcribing RNA into cDNA are well known and described in Sambrook et al., 1989. Alternative methods for reverse transcription utilize thermostable DNA polymerases.

In Vivo Diagnosis

Methods of in vivo diagnostic imaging with labeled peptides or antibodies are well-known. For example, in the technique of immunoscintigraphy, ligands or antibodies are labeled with a gamma-emitting radioisotope and introduced into a patient. A gamma camera is used to detect the location and distribution of gamma-emitting radioisotopes. See, for example, Srivastava (ed.), RADIOLABELED MONOCLONAL ANTIBODIES FOR IMAGING AND THERAPY (Plenum Press 1988), Chase, “Medical Applications of Radioisotopes,” in REMINGTON'S PHARMACEUTICAL SCIENCES, 18th Edition, Gennaro et al. (eds.), pp. 624-652 (Mack Publishing Co., 1990), and Brown, “Clinical Use of Monoclonal Antibodies,” in BIOTECHNOLOGY AND PHARMACY 227-49, Pezzuto et al. (eds.) (Chapman & Hall 1993). Also preferred is the use of positron-emitting radionuclides (PET isotopes), such as fluorine-18 (¹⁸F), gallium-68 (⁶⁸Ga), and iodine-124 (¹²⁴I). Such imaging can be conducted by direct labeling of the ligand, or by a pretargeted imaging method, see U.S. Patent Publication Nos. 20050002945, 20040018557, 20030148409 and 20050014207.

### Pharmaceutical Compositions

In some embodiments, one or more inhibitors or activators may be administered to a subject with a disease. Such agents may be administered in the form of pharmaceutical compositions. Generally, this will entail preparing compositions that are essentially free of impurities that could be harmful to humans or animals.

One generally will employ appropriate salts and buffers to render therapeutic agents stable and allow for uptake by target cells. Aqueous compositions may comprise an effective amount of an inhibitor or activator, dissolved or dispersed in a pharmaceutically acceptable carrier or aqueous medium. The phrase “pharmaceutically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical forms suitable for use include sterile aqueous solutions or dispersions and sterile powders for the preparation of sterile solutions or dispersions. It must be stable under the conditions of manufacture and storage and must be preserved against the contaminating action of microorganisms, such as bacteria and fungi.

In certain embodiments, an effective amount of a therapeutic agent must be administered to the subject. An “effective amount” is the amount of the agent that produces a desired effect. An effective amount will depend, for example, on the efficacy of the agent and on the intended effect. An effective amount of a particular agent for a specific purpose can be determined using methods well known to those in the art.

Pharmaceutically Acceptable Carriers

As used herein, “pharmaceutically acceptable carrier” includes any and all solvents, dispersion media, coatings, antibacterial and antifungal agents, isotonic and absorption delaying agents, and the like that are physiologically compatible. In particular embodiments, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the presence of microorganisms may be ensured both by sterilization procedures and by the inclusion of various antibacterial and antifungal agents, for example, paraben, chlorobutanol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

A “pharmaceutically acceptable salt” refers to a salt that retains the desired biological activity of the parent compound and does not impart any undesired toxicological effects (see e.g., Berge, et al., J. Pharm. Sci. 66:1-19 (1977)). Examples of such salts include acid addition salts and base addition salts.

The pharmaceutical compositions of the present invention may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetraacetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the specific antibody.

Therapeutically Effective Dosages

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the therapeutic agent is being used, the route of administration, and the size (body weight, body surface or organ size) and condition (the age and general health) of the patient. Accordingly, the clinician may titer the dosage and modify the route of administration to obtain the optimal therapeutic effect.

A therapeutically effective amount is typically an amount such that when administered in a physiologically tolerable composition is sufficient to achieve a plasma of, for example, from about 0.01 μg/ml to about 300 μg/ml. In another embodiment, the concentration may be from about 1 μg/ml to about 300 μg/ml. In yet another embodiment, the concentration may be from about 1 μg/ml to about 75 μg/ml. In yet another embodiment, the concentration may be from about 15 μg/ml to about 50 μg/ml. Dosages may, of course, vary according to frequency and duration of administration.

For any compound, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans.

The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

A physician or veterinarian having ordinary skill in the art can readily determine and prescribe the effective amount of the pharmaceutical composition required. For example, the physician or veterinarian could start doses of the compounds employed in the pharmaceutical composition at levels lower than that required in order to achieve the desired therapeutic effect and gradually increase the dosage until the desired effect is achieved. In general, a suitable daily dose of a composition will be that amount of the compound which is the lowest dose effective to produce a therapeutic effect.

Routes of Administration

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, intraperitoneal, intranasal, enteral, topical, sublingual, urethral, vaginal, or rectal means, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use pharmaceutical compositions in an ex vivo manner. In such instances, cells, tissues, or organs that have been removed from the patient are exposed to the pharmaceutical compositions after which the cells, tissues and/or organs are subsequently implanted back into the patient.

Therapeutic compositions can be administered with medical devices known in the art. For example, in a preferred embodiment, a therapeutic composition of the invention can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In another embodiment, a pharmaceutical composition may be formulated for inhalation. For example, a binding agent may be formulated as a dry powder for inhalation. Polypeptide or nucleic acid molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

Additional pharmaceutical compositions will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 that describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate [Sidman et al., Biopolymers 22:547-556 (1983)], poly(2-hydroxyethyl-methacrylate) [Langer et al., J. Biomed. Mater. Res. 15:167-277, (1981)] and [Langer et al., Chem. Tech. 12:98-105 (1982)], ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., Proc. Natl. Acad. Sci. (USA) 82:3688-3692 (1985); EP 36,676; EP 88,046; EP 143,949.

### Peptide Administration

Various embodiments of the claimed methods and/or compositions may concern one or more therapeutic peptides to be administered to a subject. Administration may occur by any route known in the art. In certain embodiments, oral administration is contemplated.

Unmodified peptides administered orally to a subject can be degraded in the digestive tract and depending on sequence and structure may exhibit poor absorption across the intestinal lining. However, methods for chemically modifying peptides to render them less susceptible to degradation by endogenous proteases or more absorbable through the alimentary tract are known (see, for example, Blondelle et al., 1995, Biophys. J. 69:604-11; Ecker and Crooke, 1995, Biotechnology 13:351-69; Goodman and Ro, 1995, BURGER'S MEDICINAL CHEMISTRY AND DRUG DISCOVERY, VOL. 1, ed. Wollf, John Wiley & Sons; Goodman and Shao, 1996, Pure & Appl. Chem. 68:1303-08). Methods for preparing libraries of peptide analogs, such as peptides containing D-amino acids; peptidomimetics consisting of organic molecules that mimic the structure of a peptide; or peptoids such as vinylogous peptoids, have also been described and may be used to construct therapeutic peptides suitable for oral administration to a subject.

In certain embodiments, preparation and administration of peptide mimetics that mimic the structure of any selected peptide may be used within the scope of the claimed methods and compositions. In such compounds, the standard peptide bond linkage may be replaced by one or more alternative linking groups, such as CH₂—NH, CH₂—S, CH₂—CH₂, CH═CH, CO—CH₂, CHOH—CH₂ and the like. Methods for preparing peptide mimetics are well known (for example, Hruby, 1982, Life Sci 31:189-99; Holladay et al., 1983, Tetrahedron Lett. 24:4401-04; Jennings-White et al., 1982, Tetrahedron Lett. 23:2533; Almquiest et al., 1980, J. Med. Chem. 23:1392-98; Hudson et al., 1979, Int. J. Pept. Res. 14:177-185; Spatola et al., 1986, Life Sci 38:1243-49; U.S. Pat. Nos. 5,169,862; 5,539,085; 5,576,423, 5,051,448, 5,559,103, each incorporated herein by reference.) Peptide mimetics may exhibit enhanced stability and/or absorption in vivo compared to their peptide analogs.

Alternatively, therapeutic peptides may be administered by oral delivery using N-terminal and/or C-terminal capping to prevent exopeptidase activity. For example, the C-terminus may be capped using amide peptides and the N-terminus may be capped by acetylation of the peptide. Peptides may also be cyclized to block exopeptidases, for example by formation of cyclic amides, disulfides, ethers, sulfides and the like.

Peptide stabilization may also occur by substitution of D-amino acids for naturally occurring L-amino acids, particularly at locations where endopeptidases are known to act. Endopeptidase binding and cleavage sequences are known in the art and methods for making and using peptides incorporating D-amino acids have been described (e.g., U.S. Patent Application Publication No. 20050025709). The skilled artisan will be aware that peptide modification should be followed by testing for target binding activity to direct the course of peptide modification. In certain embodiments, peptides and/or proteins may be orally administered by co-formulation with proteinase- and/or peptidase-inhibitors.

Other methods for oral delivery of therapeutic peptides are disclosed in Mehta (“Oral delivery and recombinant production of peptide hormones,” June 2004, BioPharm International). The peptides are administered in an enteric-coated solid dosage form with excipients that modulate intestinal proteolytic activity and enhance peptide transport across the intestinal wall. Relative bioavailability of intact peptides using this technique ranged from 1% to 10% of the administered dosage. Insulin has been administered in dogs using enteric-coated microcapsules with sodium cholate and a protease inhibitor (Ziv et al., 1994, J. Bone Miner. Res. 18 (Suppl. 2):792-94). Oral administration of peptides has been performed using acylcarnitine as a permeation enhancer and an enteric coating (Eudragit L30D-55, Rohm Pharma Polymers, see Mehta, 2004). Excipients of use for orally administered peptides may generally include one or more inhibitors of intestinal proteases/peptidases along with detergents or other agents to improve solubility or absorption of the peptide, which may be packaged within an enteric-coated capsule or tablet (Mehta, 2004). The enteric coating is resistant to acid, allowing the peptide to pass through the stomach into the intestine for absorption. Organic acids may be included in the capsule to acidify the intestine and inhibit intestinal protease activity once the capsule dissolves in the intestine (Mehta, 2004). Another alternative for oral delivery of peptides would include conjugation to polyethylene glycol (PEG)-based amphiphilic oligomers, increasing absorption and resistance to enzymatic degradation (Soltero and Ekwuribe, 2001, Pharm. Technol. 6:110).

In alternative embodiments, therapeutic peptides may be administered by an inhalational route (e.g., Sievers et al., 2001, Pure Appl. Chem. 73:1299-1303). Supercritical carbon dioxide aerosolization has been used to generate nano or micro-scale particles out of a variety of pharmaceutical agents, including proteins and peptides (Id.) Microbubbles formed by mixing supercritical carbon dioxide with aqueous protein or peptide solutions may be dried at lower temperatures (25 to 65° C.) than alternative methods of pharmaceutical powder formation, retaining the structure and activity of the therapeutic peptide (Id.) In some cases, stabilizing compounds such as trehalose, sucrose, other sugars, buffers or surfactants may be added to the solution to further preserve functional activity. The particles generated are sufficiently small to be administered by inhalation, avoiding some of the issues with intestinal proteases/peptidases and absorption across the gastrointestinal lining.

### Combination Therapies and Putative Inhibitors/Activators

In some embodiments, the inhibitors or activators of hibernation-related genes or their protein products may be administered with one or more known therapeutic agents, such as immunomodulators, cytokines, chemokines, lymphokines, chemotherapeutic agents, growth factors, anti-inflammatory agents, IL-1 inhibitors, small molecules, anti-rheumatic drugs, kinase inhibitors and other known therapeutic agents. Such known therapeutic agents may also be candidate activators or inhibitors of hibernation-related genes or protein products, which may be assayed for their effects using known methods as discussed above.

Immunomodulators

As used herein, the term “immunomodulator” includes cytokines, stem cell growth factors, lymphotoxins and hematopoietic factors, such as interleukins, colony stimulating factors and interferons (e.g., interferons-α, -β and -γ). Exemplary immunomodulators include IL-2, IL-6, IL-10, IL-12, IL-18, IL-21, interferon-gamma, TNF-alpha, and the like.

The term “cytokine” is a generic term for proteins or peptides released by one cell population which act on another cell as intercellular mediators. As used herein, examples of cytokines include lymphokines, monokines, growth factors and traditional polypeptide hormones. Included among the cytokines are growth hormones such as human growth hormone, N-methionyl human growth hormone, bovine growth hormone; parathyroid hormone; thyroxine; insulin; proinsulin; relaxin; prorelaxin; glycoprotein hormones such as follicle stimulating hormone (FSH), thyroid stimulating hormone (TSH), luteinizing hormone (LH); hepatic growth factor; prostaglandin, fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, LIF, erythropoietin (EPO), kit-ligand or FLT-3, angiostatin, thrombospondin, endostatin, tumor necrosis factor and LT.

Chemokines generally act as chemoattractants to recruit immune effector cells to the site of chemokine expression. Chemokines include, but are not limited to, RANTES, MCAF, MIP1-alpha, MIP1-Beta, and IP-10.

Chemotherapeutic Agents

Chemotherapeutic agents include, for example, alkylating agents such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as triethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinium coordination complexes such as cisplatin and carboplatin, anthracenediones such as mitoxantrone, substituted urea such as hydroxyurea, methylhydrazine derivatives including N-methylhydrazine (M1H) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestins such as hydroxyprogesterone caproate, medroxyprogesterone acetate and megestrol acetate; estrogens such as diethylstilbestrol and ethinyl estradiol equivalents; antiestrogens such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

IL-1 Inhibitors

Inhibitors of IL-1 include, but are not limited to, receptor-binding peptide fragments of IL-1, antibodies directed against IL-1 or IL-1 beta or IL-1 receptor type I, and recombinant proteins comprising all or portions of receptors for IL-1 or modified variants thereof, including genetically-modified muteins, multimeric forms and sustained-release formulations. Specific antagonists include IL-Ira polypeptides, IL-1 beta converting enzyme (ICE) inhibitors, antagonistic type I IL-1 receptor antibodies, IL-1 binding forms of type I IL-1 receptor and type II IL-1 receptor, antibodies to IL-1, including IL-1 alpha and IL-1 beta and other IL-1 family members, and a therapeutic known as IL-1 Trap (Regeneron). IL-Ira polypeptides include the forms of IL-Ira described in U.S. Pat. No. 5,075,222 and modified forms and variants including those described in U.S. Pat. No. 5,922,573, WO 91/17184, WO 92 16221, and WO 96 09323. IL-1 beta converting enzyme (ICE) inhibitors include peptidyl and small molecule ICE inhibitors including those described in PCT patent applications WO 91/15577; WO 93/05071; WO 93/09135; WO 93/14777 and WO 93/16710; and European patent application 0 547 699. Non-peptidyl compounds include those described in PCT patent application WO 95/26958, U.S. Pat. No. 5,552,400, U.S. Pat. No. 6,121,266, and Dolle et al., J. Med. Chem., 39, pp. 2438-2440 (1996). Additional ICE inhibitors are described in U.S. Pat. Nos. 6,162,790, 6,204,261, 6,136,787, 6,103,711, 6,025,147, 6,008,217, 5,973,111, 5,874,424, 5,847,135, 5,843,904, 5,756,466, 5,656,627, 5,716,929. IL-1 binding forms of Type I IL-1 receptor and type II IL-1 receptor are described in U.S. Pat. Nos. 4,968,607, 4,968,607, 5,081,228, Re 35,450, 5,319,071, and 5,350,683. Other suitable IL-1 antagonists include, but are not limited to, peptides derived from IL-1 that are capable of binding competitively to the IL-1 signaling receptor, IL-1 R type I. Additional guidance regarding certain IL-1 (and other cytokine) antagonists can be found in U.S. Pat. No. 6,472,179.

Miscellaneous

Further suitable compounds include, but are not limited to, small molecules such as thalidomide or thalidomide analogs, pentoxifylline, or matrix metalloproteinase (MMP) inhibitors or other small molecules. Suitable MMP inhibitors for this purpose include, for example, those described in U.S. Pat. Nos. 5,883,131, 5,863,949 and 5,861,510 as well as mercapto alkyl peptidyl compounds as described in U.S. Pat. No. 5,872,146. Other small molecules capable of reducing TNF-alpha production, include, for example, the molecules described in U.S. Pat. Nos. 5,508,300, 5,596,013, and 5,563,143. Additional suitable small molecules include, but are not limited to, MMP inhibitors as described in U.S. Pat. Nos. 5,747,514, and 5,691,382, as well as hydroxamic acid derivatives such as those described in U.S. Pat. No. 5,821,262. Further suitable molecules include, for example, small molecules that inhibit phosphodiesterase IV and TNF-alpha production, such as substituted oxime derivatives (WO 96/00215), quinoline sulfonamides (U.S. Pat. No. 5,834,485), aryl furan derivatives (WO 99/18095) and heterobicyclic derivatives (WO 96/01825; GB 2 291 422 A). Also useful are thiazole derivatives that suppress TNF-alpha and IFN-gamma (WO 99/15524), as well as xanthine derivatives that suppress TNF-alpha and other proinflammatory cytokines (see, for example, U.S. Pat. Nos. 5,118,500, 5,096,906 and 5,196,430). Additional small molecules that may be of use for treating the herein described conditions include those disclosed in U.S. Pat. Nos. 5,336,503; 5,547,979; 5,618,809; 5,945,440; 6,432,751 and 6,696,480.

Anti-Inflammatory Agents

Further examples of drugs and drug types which can be administered by combination therapy include, but are not limited to, antivirals, antibiotics, analgesics (e.g., acetaminophen, codeine, propoxyphene napsylate, oxycodone hydrochloride, hydrocodone bitartrate, tramadol), corticosteroids, antagonists of inflammatory cytokines, Disease-Modifying Anti-Rheumatic Drugs (DMARDs), Non-Steroidal Anti-Inflammatory drugs (NSAIDs), and Slow-Acting Anti-Rheumatic Drugs (SAARDs).

Exemplary Disease-Modifying Anti-Rheumatic Drugs (DMARDS) include, but are not limited to: Rheumatrex™ (methotrexate); Enbrel® (etanercept); Remicade® (inflixiantibody); Humira™ (adalimuantibody); Segard® (afelimoantibody); Arava™ (leflunomide); Kineret™ (anakinra); Arava™ (leflunomide); D-penicillamine; Myochrysine; Plaquenil; Ridaura™ (auranofin); Solganal; lenercept (Hoffman-La Roche); CDP870 (Celltech); CDP571 (Celltech), as well as the antibodies described in EP 0 516 785 B1, U.S. Pat. No. 5,656,272, EP 0 492 448 A1; onercept (Serono; CAS reg. no. 199685-57-9); MRA (Chugai); Imuran™ (azathioprine); NFKB inhibitors; Cytoxan™ (cyclophosphamide); cyclosporine; hydroxychloroquine sulfate; minocycline; sulfasalazine; and gold compounds such as oral gold, gold sodium thiomalate and aurothioglucose.

The Non-Steroidal Anti-Inflammatory Drugs (NSAIDs) owe their anti-inflammatory action, at least in part, to the inhibition of prostaglandin synthesis. Goodman and Gilman, The Pharmacological Basis of Therapeutics, MacMillan 7^{th} Edition (1985). Examples of NSAIDs include, but are not limited to: Anaprox™, Anaprox DS™ (naproxen sodium); Ansaid™ (flurbiprofen); Arthrotec™ (diclofenac sodium+misoprostil); Cataflam™/Voltaren™ (diclofenac potassium); Clinoril™ (sulindac); Daypro™ (oxaprozin); Disalcid™ (salsalate); Dolobid™ (diflunisal); EC Naprosyn™ (naproxen sodium); Feldene™ (piroxicam); Indocim™, Indocin SR™ (indomethacin); Lodine™, Lodine XL™ (etodolac); Motrin™ (ibuprofen); Naprelan™ (naproxen); Naprosyn™ (naproxen); Orudis™, (ketoprofen); Oruvail™ (ketoprofen); Relafen™ (nabumetone); Tolectin™, (tolmetin sodium); Trilisate™ (choline magnesium trisalicylate); Cox-1 inhibitors; Cox-2 Inhibitors such as Vioxx™ (rofecoxib); Arcoxia™ (etoricoxib), Celebrex™ (celecoxib); Mobic™ (meloxicam); Bextra™ (valdecoxib), Dynastat™ paracoxib sodium; Prexige™ (lumiracoxib), and nambumetone. Additional suitable NSAIDs, include, but are not limited to, the following: .epsilon.-acetamidocaproic acid, S-adenosylmethionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixim, lefetamine HCl, leflunomide, lofemizole, lotifazole, lysin clonixinate, meseclazone, nabumetone, nictindole, nimesulide, orgotein, orpanoxin, oxaceprolm, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, A177B, AP504, AU8001, BPPC, BW540C, CHINOIN 127, CN100, EB382, EL508, F1044, FK-506, GV3658, ITF182, KCNTEI6090, KME4, LA2851, MR714, MR897, MY309, ONO.sub.3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770.

Suitable SAARDs or DMARDS include, but are not limited to: allocupreide sodium, auranofin, aurothioglucose, aurothioglycamide, azathioprine, brequinar sodium, bucillamine, calcium 3-aurothio-2-propanol-1-sulfonate, chlorambucil, chloroquine, clobuzarit, cuproxoline, cyclophosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine.

Inhibitors of kinases in signaling cascades may also be suitable agents. These include, but are not limited to, agents which are capable of inhibiting P-38 (a.k.a., “RK” or “SAPK-2”, Lee et al., Nature, 372:739 (1994). P-38 is described as a serine/threonine kinase (see Han et al., Biochimica Biophysica Acta, 1265:224-227 (1995). Inhibitors of P-38 have been shown to intervene between the extracellular stimulus and the secretion of IL-1 and TNF-alpha from the cell involves blocking signal transduction through inhibition of a kinase which lies on the signal pathway.

Additionally suitable are MK2 inhibitors, and tpl-2 inhibitors. Additionally, T-cell inhibitors are also suitable, including, for example, ctla-4, CsA, Fk-506, OX40, OX40R-Fc, OX40 antibody, OX40 ligand, OX40 ligand antibody, lck, and ZAP70. Also suitable are retinoids, including oral retinoids, as well as antagonists of TGF-beta.

Further suitable agents may include, for example, any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate and sulfasalazine. Structurally related salicylic acid derivatives having similar analgesic and anti-inflammatory properties are also intended to be encompassed by this group. Additionally suitable agents include, for example propionic acid derivatives, prodrug esters or pharmaceutically acceptable salts thereof. The propionic acid derivatives, prodrug esters and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group. Also suitable for use are acetic acid derivatives, prodrug esters or pharmaceutically acceptable salts thereof. The acetic acid derivatives, prodrug esters and pharmaceutically acceptable salts thereof comprise: acemetacin, alclofenac, amfenac, bufexamac, cinmetacin, clopirac, delmetacin, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, zidometacin and zomepirac. Structurally related acetic acid derivatives having similar analgesic and anti-inflammatory properties are also intended to be encompassed by this group. Further suitable for use as described herein are fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefanamic acid, niflumic acid, talniflumate, terofenamate, tolfenamic acid and ufenamate. Structurally related fenamic acid derivatives having similar analgesic and anti-inflammatory properties are also intended to be encompassed by this group.

Additional suitable compounds include, but are not limited to: BN 50730; tenidap; E 5531; tiapafant PCA 4248; nimesulide; panavir; rolipram; RP 73401; peptide T; MDL 201,449A; (1R,3S)-Cis-1-[9-(2,6-diamin-opurinyl)]-3-hydroxy-4-cyclopentene hydrochloride; (1R,3R)-trans-1-[9-(2,6-diamino)purine]-3-acetoxycyclopentane; (1R,3R)-trans-1-[9-adenyl)-3-azido-cyclopentane hydrochloride and (1R,3R)-trans-1-[6-hydroxy-purin-9-yl)-3-az-idocyclopentane.

Anti-Angiogenic Agents

Pharmaceutical compositions can include one or more anti-angiogenic agents selected from the group consisting of antagonists of Ang-1 and hibernation-related genes or their expressed proteins (and their receptors), VEGF (Avastin, VEGF-TRAP, etc.), VEGF receptors, and IL-8, B-FGF, and small molecule inhibitors of KDR and other mediators of inflammation. Inhibitors of inflammation include such compounds as: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol, (EntreMed, USA); TLC ELL-12, (Elan, Ireland); anecortave acetate, (Alcon, USA); alpha-D148 antibody, (Amgen, USA); CEP-7055, (Cephalon, USA); anti-Vn antibody, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland, EP 970070); ARGENT technology, (Ariad, USA); YIGSR-Stealth, (Johnson & Johnson, USA); fibrinogen-E fragment, (BioActa, UK); inflammation inhibitor, (Trigen, UK); TBC-1635, (Encysive Pharmaceuticals, USA); SC-236, (Pfizer, USA); ABT-567, (Abbott, USA); Metastatin, (EntreMed, USA); inflammation inhibitor, (Tripep, Sweden); maspin, (Sosei, Japan); 2-methoxyestradiol, (Oncology Sciences Corporation, USA); ER-68203-00, (IVAX, USA); Benefin, (Lane Labs, USA); Tz-93, (Tsumura, Japan); TAN-1120, (Takeda, Japan); FR-111142, (Fujisawa, Japan, JP 02233610); platelet factor 4, (RepliGen, USA, EP 407122); vascular endothelial growth factor antagonist, (Borean, Denmark); cancer therapy, (University of South Carolina, USA); bevacizuantibody (pINN), (Genentech, USA); inflammation inhibitors, (SUGEN, USA); XL 784, (Exelixis, USA); XL 647, (Exelixis, USA); antibody, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); inflammation inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuxiantibody (INN), (Aventis, France); AVE 8062, (Ajinomoto, Japan); AS1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, inflammation, (EntreMed, USA); urokinase plasminogen activator inhibitor, (Dendreon, USA); oglufanide (pINN), (Melmotte, USA); HIF-1 alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Childrens Hospital, USA and EntreMed, USA); antibody, KDR, (ImClone Systems, USA); antibody, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrug, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); antibody, vascular endothelium growth factor, (Xenova, UK); irsogladine (INN), (Nippon Shinyaku, Japan); RG 13577, (Aventis, France); WX 360, (Wilex, Germany); squalamine (pINN), (Genaera, USA); RPI 4610, (Sirna, USA); cancer therapy, (Marinova, Australia); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); thrombospondin 1 inhibitor, (Allegheny Health, Education and Research Foundation, USA).

### Kits

Various embodiments may concern kits containing components suitable for treating or diagnosing diseased tissue in a patient, such as inhibitors or activators of hibernation-related genes or their protein products or antibodies or other binding agents that bind to hibernation-related mRNAs or proteins.

If the composition containing components for administration is not formulated for delivery via the alimentary canal, such as by oral delivery, a device capable of delivering the kit components through some other route may be included. One type of device, for applications such as parenteral delivery, is a syringe that is used to inject the composition into the body of a subject. Inhalation devices may also be used.

The kit components may be packaged together or separated into two or more separate containers. In some embodiments, the containers may be vials that contain sterile, lyophilized formulations of a composition that are suitable for reconstitution. A kit may also contain one or more buffers suitable for reconsititution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

### EXAMPLES

The following examples are included to demonstrate preferred embodiments of the invention. It should be appreciated by those of skill in the art that the techniques disclosed in the examples which follow represent techniques discovered to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods for Identifying Hibernation-Related Genes

Disclosed herein is a new gene expression profiling technology, using oligonucleotide beadarrays manufactured by Illumina Corporation (Kuhn et al. 2004). This technology involves the preparation of pooled libraries of 3 μm beads. Each bead is covalently attached with >10⁵ copies of identical oligonucleotide probes. The beads self-assemble into the etched wells on the surface of the beadarray with one bead per well. Each beadarray can support 50,000 beads, representing 1,500 unique probes with 30-fold redundancy for each probe. Each oligonucleotide probe is concatenated by a 23-mer address sequence that is used to decode the exact location of the probe on the beadarray during the decoding process (Gunderson et al. 2004) and a 50-mer gene-specific sequence that is used to hybridize with the fluorescently labeled RNA sequences. 700 genes can be represented on a customized beadarray with two probes designed for each gene. As a result of the high redundancy built into the beadarray, it exhibits high selectivity and sensitivity in gene expression profiling (Kuhn et al. 2004). We customized two formats of beadarrays, referred to herein as 1A and 2A, covering about 1,400 total genes with probes designed from available ground squirrel mRNA sequences. Two types of high-throughput beadarray platforms were used—a 16-sample beadchip and a 96-sample array matrix. The high throughput design of these two beadarray platforms enabled us to include a wide range of tissues (BAT, liver, heart, hypothalamus, and skeletal muscle) and multiple stages of hibernation (early arousal, late arousal, early torpor, and late torpor). The candidate genes identified on the beadarrays and other genes in important pathways were then extensively tested by real-time PCR assay.

Animals

Arctic ground squirrels (S. parryii kenocottii) were trapped during July on the North Slope of Alaska near Toolik Lake (68° N. 149° W, elevation 809 m) and transported to the University of Alaska, Fairbanks. Animals were housed at 18±2° C. with a 16:8-h light:dark photoperiod and provided with Mazuri Rodent Chow and water ad libitum, with supplements of sunflower seeds, carrots, and apple slices. Animals were transferred in September to 5±1° C. with a 4:20-h photoperiod where they entered hibernation. A pilot study using 16-sample beadchip compared torpid animals (n=9) with non-hibernating post-reproductive animals (n=7). Torpid animals were monitored using the traditional saw-dust method, i.e., they were inspected twice daily and wood shavings were placed on the dorsal surface of torpid animals to assess by their presence or absence the duration of torpor bouts and the occurrence of arousal episodes. Animals in torpor were sampled for tissues after no fewer than five days of continuous torpor in at least the third torpor bout of the winter hibernation season. Post-reproductive animals had spontaneously ended hibernation while remaining in the same environmental conditions, completed reproductive maturation and regression as assessed by external inspection of gonads and genitalia (Barnes, et al., 1986) and had entered molt.

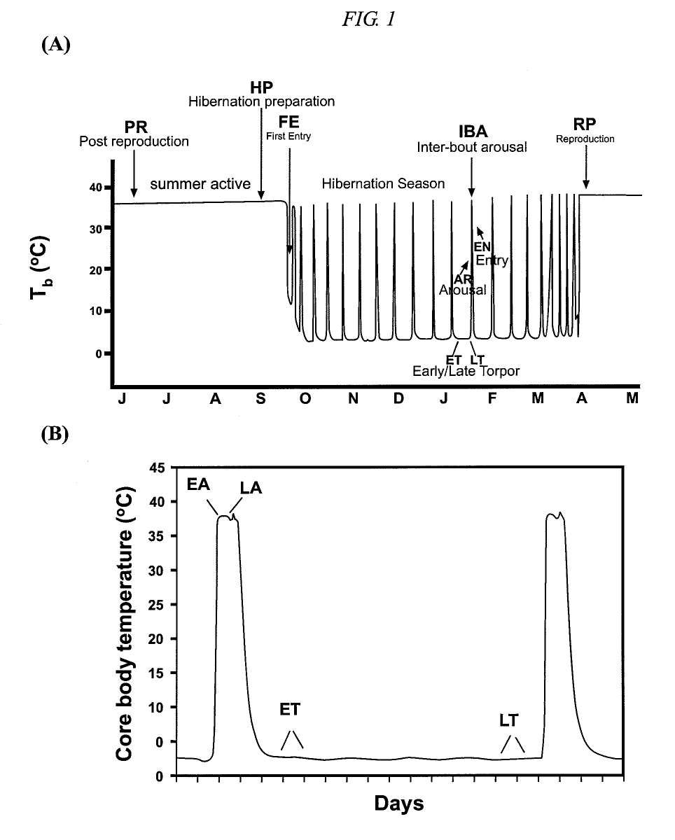

Hibernating animals used in 96-sample array matrix experiment were housed at 5±1° C. with a 4:20-h photoperiod and monitored for precise stages of torpor and arousal by an automated telemetry system that recorded core body temperatures (T_{b}) every 10 minutes, as indicated by temperature-sensitive transmitters that were implanted in the abdominal regions of the animals (Buck and Barnes, 2000). Animals sampled in four states during hibernation included: animals sampled early in a torpor bout after 10-20% of the duration of the previous torpid bout (ET, n=4); animals sampled late in a torpor bout after 80-90% of the duration of the previous torpor bout (LT, n=5); animals sampled early after spontaneously arousing 1-2 hours after their T_{b} had increased above 30° C. during rewarming (EA, n=4); and animals sampled later in the arousal episode 7-8 hrs after T_{b} had increased above 30° C. (LA, n=4). The four states during hibernation in a telemetered animal are illustrated in FIG. 1.

In real-time PCR assays, we added eight more (two more for hypothalamus) telemetered animals to the 24 animals used in array matrix experiments. A total of 32 (26 for hypothalamus) animals including early aroused animals (n=6, n=5 for hypothalamus); late aroused animals (n=7, n=5 for hypothalamus); early torpid animals (n=4); late torpid animals (n=7, n=5 for hypothalamus); and post-reproductive animals (n=8, n=7 for hypothalamus) were used in real-time PCR assay. Animals sampled during arousal episodes had T_{b} of 35-37° C., and animals sampled during torpor had T_{b} of 5-7° C., as indicated by telemetry. Torpid animals were euthanized by decapitation without anesthesia. Aroused and post-reproductive animals were deeply anesthetized with isoflurane and decapitated. Brown adipose tissue, liver, heart, hypothalamus, and skeletal muscle were rapidly dissected, frozen in liquid nitrogen, and stored at −80° C. until total RNA was isolated at a later date.

Sample Preparation and Hybridization

Total RNA was prepared from frozen tissues by homogenizing directly in liquid nitrogen using RNeasy kits (Qiagen) with mortar and pestle. Tissues from heart and skeletal muscle were treated by proteinase K digestion to remove the connective tissues prior to the RNA extraction. RNA was processed by DNase I treatment and RNA quality was assessed by 1.2% formaldehyde agarose gel electrophoresis under the denaturing condition using ethidium bromide (EtBr) post staining. The density of total RNA of each sample was measured by spectrometer. 100 ng of each total RNA sample was subsequently linearly amplified with Ambion Illumina RNA Amplification kits (Ambion) using a modified T7 Eberwine procedure (Van Gelder et al. 1990). Biotin-16-UTP (Enzo) was used during the in vitro transcription. All samples of the same tissue were amplified in the same batch. Labeled amplified RNA (1 μg per array on 96-sample array matrix and 500 ng per array on 16-sample beadchip) was hybridized onto each array and incubated at 55° C. for 16 hrs followed by washing and blocking steps according to manufacturer's instruction. Streptavidin-Cy3 (Amersham Biosciences) was used to cross-link Cy3 with biotin labeled amplified RNA. The arrays were scanned using an Illumina Bead Array Reader scanner according to manufacturer's instruction. Array data was processed and analyzed by Illumina BeadStudio software.

Beadarray Probe Design.

Beadarray probes were designed from non-redundant high quality cDNA sequences of three closely-related ground squirrel species: Spermophilus lateralis, Spermophilus parryii, and Spermophilus tridecemlineatus. S. lateralis and S. tridecemlineatus share on average 99% mRNA sequence identities with S. parryii at the nucleotide level (Yan et al. 2006). S. lateralis sequences were downloaded from squirrelBASE 2.0 (Nov. 10, 2003) of Laboratory for Environmental Gene Regulation (LEGR) Data Centre at Liverpool University. Among 5,109 sub-groups (EST clusters) in squirrelBASE 2.0, only the 1,846 sub-groups aligned with SwissProt were used in this study. We also downloaded the annotation and alignment information for the SwissProt aligned sub-groups.

To guarantee the quality and non-redundancy of the sequences, we further processed them according to the following procedures: 1. Sequences outside the SwissProt alignments were trimmed to avoid vector contaminations and sequencing errors. 2. If there are more than one sub-groups belonging to the same group (EST clusters with a less stringent condition), we only kept the sub-group with the longest SwissProt alignment in that group. 3. All sequences were repeat-masked by RepeatMaser (Smit et al. 1996) and the sequences with unmasked nucleotides less than 150 bps were removed. 4. The sequences annotated as hypothetical proteins were removed. 5. For the sequences aligned in the reverse complement direction, their reverse complements were used.

After the preliminary processing, 1,545 S. lateralis sequences remained. Among them, 1,329 sequences were actually used in the beadarray probes design. 81 S. parryii genes (GenBank accessions: DQ333962-DQ334051) were sequenced from our previous study (Yan et al. 2006). These S. parryii sequences were aligned against the S. lateralis sequences using the blastn program (Altschul et al. 1990) to identify those that already existed in the S. lateralis sequences. After removing the redundant sequences, we obtained 62 S. parryii sequences for probes design. In addition, 16 non-redundant S. tridecemlineatus sequences downloaded from GenBank were also used in probe design.

Seven genes: actin beta (Actb), eukaryotic translation elongation factor 1 alpha 1 (Eef1a1), glyceraldehyde-3-phosphate dehydrogenase (Gapd), ribosomal protein S9 (Rps9), tubulin beta 2B (Tubb2b), ribosomal protein S3 (Rps3), and ubiquitin C (Ubc) were chosen as house-keeping genes to be present on both 1A and 2A arrays. Overall, 1,407 ground squirrel sequences were sent to Illumina Corp. for probe design. Two 50 bp probes were designed for every gene except for three genes on the 2A array: heat shock 10 kDa protein 1 (Hspe1), major histocompatibility complex, class II, DP beta 1 (Hla-dpb1), and 1-acylglycerol-3-phosphate O-acyltransferase 3 (Agpat3) with only one 50 bps probe designed.

The sequence sources of the genes on 1A and 2A arrays are shown in Table 1. To obtain the standard gene names and symbols for the 1,407 ground squirrel sequences, we aligned them onto the RefSeq (Pruitt et al. 2005) sequences using blastn program (Altschul et al. 1990). The RefSeq sequence with the highest blast score was identified to be the homologous sequence for each ground squirrel sequence. The accession numbers of homologous RefSeq sequences were then up-loaded to Stanford Source (Stanford University) to obtain the gene names and symbols.

Data Analysis

The array data was background subtracted and normalized by rank-invariant method using Illumina BeadStudio software. The detection score (detection probability between 0 and 1) of each gene on every array was also obtained from Illumina BeadStudio software. For the 16-sample beadchip experiment, two-stage analysis comparing torpid animals (T) with post-reproductive animals (P) was done. For the 96-sample array matrix experiment, three-stage analysis among aroused animals (A), torpid animals (T), and post-reproductive animals (P) was done where A=EA+LA and T=ET+LT. The detection score of each gene in any stage (T and P in two-stage analysis and A, T, P in three-stage analysis) was defined as the median value of detection scores of all arrays in that stage. The detection score of a gene was defined as the maximum value of detection scores of all stages included in the analysis. We only included the genes with detection score>0.99 in the analysis. This definition of detection score allowed to include the genes only detected in one particular stage but not the other stages in our analysis. In the two-stage analysis (T and P), Welch two-sample t-test was used. In the three-stage analysis (A, T, P), one-way ANOVA followed by post hoc Tukey's test with HSD (Honestly Significant Difference) was used. All statistical analyses were done in R. All microarray data series were submitted to NCBI Gene Expression Omnibus (GEO) with accession number: GSE5414.

Real-Time PCR

A total of 303 Real-time PCR tests were conducted on the differentially expressed genes identified in three-stage analysis in BAT, liver, heart, and hypothalamus in 96-sample array matrix experiments—those identified in two-stage analysis in BAT, liver, and skeletal muscle in 16-sample beadchip experiments, and those not present on our beadarray but involved in important functional pathways. Gene-specific primers were designed based on the ground squirrel sequences pooled from S. lateralis, S. parryii, and S. tridecemlineatus using Primer Express software (Applied Biosystems).

Two-step real-time PCR was performed on an ABI-7900 HT system (Applied Biosystems) using SYBR Green reagent (Applied Biosystems). The density of total RNA of each sample was measured by spectrometer. cDNA was synthesized from 100 ng total RNA of each sample using Multiscribe reverse transcriptase (Applied Biosystems) with random hexamer primer in 10 μl reaction at 25° C. for 10 min, 48° C. for 30 min, and 95° C. for 5 min. The synthesized cDNA was 10× diluted using RNase-free water into 100 μl solution. 4 μl of diluted cDNA solution was used in each 20 μl Real-time PCR reaction. Cycle parameters were: 95° C. for 10 min hot start and 40 cycles of 95° C. for 15 s; and 60° C. for 1 min.

The 18S gene (GenBank accession: X00686) was used as an endogenous house-keeping gene for normalization. PCR product specificity was checked by melting curve analysis. The critical threshold (C_{T}) value is the PCR cycle number where the PCR growth curve crosses a defined threshold in the linear range of reaction. It can be related to gene expression values by log₂(expression value)=−ΔC_{T}, where ΔC_{T} is the difference between the critical threshold of target gene and that of 18S gene. Similar to the data analysis on the beadarrays, one-way ANOVA followed by post hoc Tukey's test was used on −ΔC_{T} in three-stage (A, T, P) analysis. In addition, four-stage analysis among Early Arousal (EA), Late Arousal (LA), Early Torpor (ET), and Late Torpor (LT) was also carried out using one-way ANOVA followed by post hoc Tukey's test.

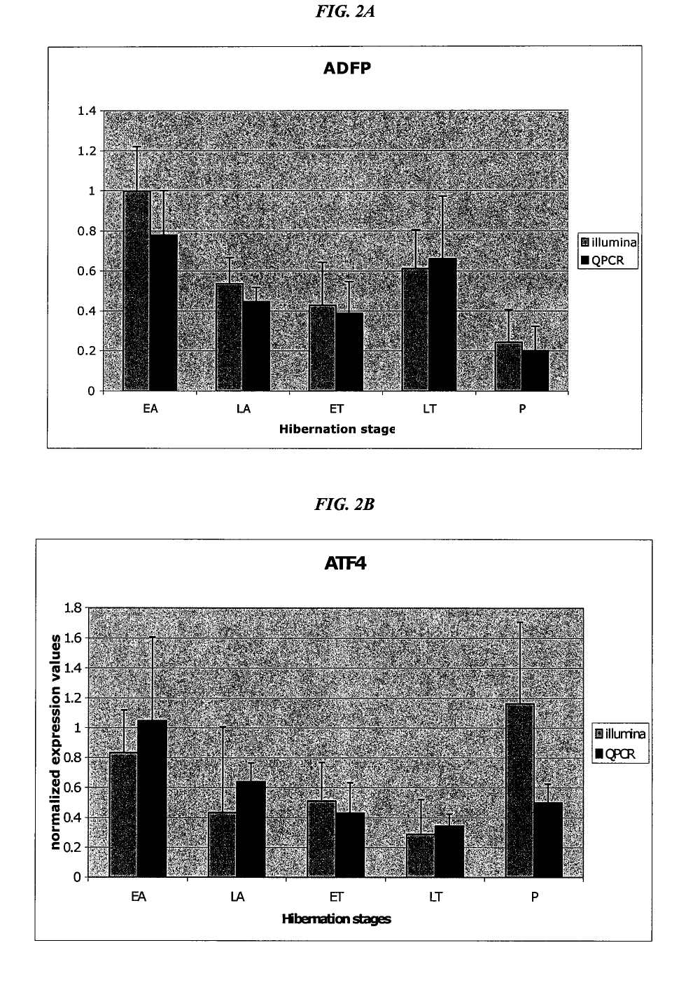

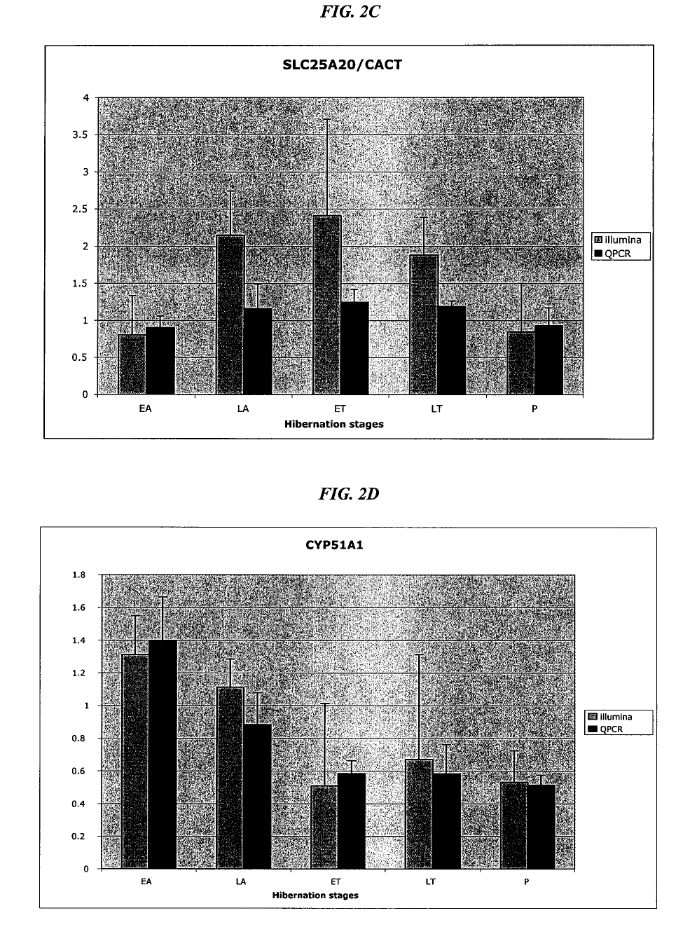

To make a more direct comparison with beadarray measurements in FIG. 2(A-D), ΔC_{T} of each sample is subtracted by the ΔC_{T} of the first early arousal animal (labeled as EA1) to obtain ΔC_{T}. Normalized expression values in real-time PCR are calculated as 2^{-ΔΔCt}. The expression value on beadarrays of each sample is also divided by that of EA 1 to obtain normalized expression value. The normalized expression values calculated for both beadarrays and real-time PCR were used to plot FIG. 2(A-D). The error bars in the figures represent the standard deviation of expression in each stage. In further studies, we first arranged the genes according to their functional categories and the animals according to their hibernation stages and then used software Cluster and TreeView (Eisen et al. 1998) to plot −ΔC_{T} for each gene and animal after median center gene adjustment without any clustering. The results of these studies showed that numbers of genes detected and the numbers of genes that were differentially expressed in different stages of the hibernation cycle (A=arousal, T=torpor, P=post-reproduction).

### Example 2

### Hibernation-Related Genes

Brown adipose tissue (BAT), liver, and skeletal muscle from torpid and post-reproductive arctic ground squirrels (AGS) were assayed on the 16-sample beadchips. Using a stringent detection criterion, a total of 317 genes were detected in at least one tissue. Two-stage analysis between torpid animals and post-reproductive animals, chosen as non-hibernating controls (NHC), was carried out by Welch's t-test. The numbers of detected and differentially expressed genes are listed in Table 2. Among the three tissues, liver shows the most detected and differentially expressed genes.

BAT, liver, heart, and hypothalamus of 24 AGS sampled early and late in a torpor bout and early and late within a spontaneous arousal episode together with the post-reproductive animals (NHC) used previously were assayed on two 96-sample Array Matrices. To compare the two beadarray platforms (96-sample array matrix and 16-sample beadchip), we also carried out two-stage analysis between torpid animals (early torpor and late torpor combined, i.e. T=ET+LT) and NHC for BAT and liver on 96-sample array matrices.

We compared the BAT and liver results on 96-sample array matrices with those on 16-sample beadchips. 20 out of 46 significant genes (P<0.05) in BAT and 28 out of 62 significant genes in liver identified on 16-sample beadchips experiment reappear as significant (P<0.05) on 96-sample array matrices. All genes showed consistent up- or down-regulation between the platforms except insulin-like growth factor 2 (Igf2) in liver which was down-regulated in the 16-sample beadchip experiment but up-regulated in 96-sample array matrix experiment. This indicates that, in spite of two different sets of torpid animals used on two different platforms, our results are consistent and repeatable in separate experiments.

Three-stage analysis between animals sampled during an arousal episode (early arousal and late arousal combined, i.e. A=EA+LA), torpid animals (early torpor and late torpor combined, i.e. T=ET+LT), NHC in BAT, liver, heart, and hypothalamus respectively on 96-sample array matrices was carried out using one-way ANOVA followed by post hoc Tukey's test. The numbers of detected and differentially expressed genes are listed in Table 3. Notably, most genes showing differential expression between aroused animals and torpid animals were down-regulated in aroused animals in BAT, liver, and heart. These genes are mainly metabolic genes such as those involved in fatty acid metabolism, amino acid metabolism, and TCA cycle. In contrast, all of six differentially expressed genes between aroused animals and torpid animals were up-regulated in aroused animals in hypothalamus.

We tested the significant genes identified in beadarray experiments together with some other genes in important functional pathways by real-time PCR assay in an enlarged sample size. We displayed the real-time PCR results in FIG. 3 according to stages of animals and functional categories of tested genes in each tissue. The same three-stage analysis was carried out on the normalized critical threshold −ΔC_{T} in real-time PCR, which corresponds to the log₂(normalized expression value) on beadarrays.

The numbers of differentially expressed genes are listed in Table 4. General agreement between beadarray experiments and real-time PCR assay were found in all tissues. In liver for example, out of 62 genes identified as significant (P<0.05) in 96-sample array matrix experiments, 42 of them also showed significant (P<0.05) differential expression in Real-time PCR assay.

Most of these genes showed consistent up- or down-regulation comparing torpid animals to NHC. Disagreement was only observed for a few genes. For example, Pck2 showed significant down-regulation in torpid animals compared to NHC on the 96-sample array matrix but showed significant up-regulation in real-time PCR. As other enzymes involved in gluconeogenesis including Pck1 and G6 pc in liver both showed consistent up-regulation in torpid animals compared to NHC in both beadarrays and real-time PCR assay, we concluded that Pck2 in liver was misclassified on the beadarrays.

Real-time PCR generally showed fewer significant differences comparing aroused animals to torpid animals than beadarray results in BAT, liver, and heart but more significant differences in hypothalamus. On the beadarrays, Alb and Slc16a12 (or Mct12) in liver were shown to be up-regulated in torpid animals compared to NHC and down-regulated in aroused animals compared to torpid animals. Real-time PCR verified their up-regulation in torpid animals compared to NHC but failed to show their down-regulation in aroused animals compared to torpid animals. Although skeletal muscle was only studied in the two-stage analysis in 16-sample beadchip experiment and not in 96-sample array matrix experiment, we still tested most of significant genes identified in 16-sample beadchip experiment using real-time PCR on the 32 samples. Excellent agreement between real-time PCR with 16-sample beadchip experiment was found when we compare torpid animals with NHC.

We represent the differential gene expression patterns in three-stage analysis by (x_{A-T}, x_{A-P}, x_{T-P}), where x_{I-J}=1 if the gene expression in stage I was significantly higher than that in stage J; −1 if significantly lower; 0 if not significantly different; I, J=A (aroused), T (torpid), P (post-reproductive or NHC). P<0.05 in post hoc Tukey's test was used as the criterion for significance. As shown in Table 5, a total of 15 different patterns are observed. The top two most abundant patterns: (0, 1, 1) with 56 cases and (0, −1, −1) with 26 cases correspond to the “seasonal” differential expression with up- or down-regulation in both aroused animals and torpid animals compared to NHC but no significant difference between aroused animals and torpid animals. Patterns (−1, 0, 1) with 6 cases and (1, 0, −1) with 1 case correspond to the “arousal-recovered” expression with up- or down-regulation during torpor compared to NHC followed by the return to the level similar to NHC during arousal. Pattern (1, 1, 0) with 7 cases corresponds to the “arousal-specific” expressions that are only up-regulated in aroused animals compared to torpid animals and NHC.

In real-time PCR assay, we had enough samples in each stage during torpor-arousal cycle to further investigate the modulation of gene expression in the multiple stages. Four-stage analysis between early arousal, late arousal, early torpor, and late torpor in all five tissues on the real-time PCR results was carried out using one-way ANOVA followed by post hoc Tukey's test. NHC was not included in this step of analysis to avoid the already identified seasonally differentially expressed genes in three-state analysis.

The numbers of significant genes were listed in Table 6. Among all step-wise comparisons between the four stages during torpor-arousal cycle, the most significant differences happen in late torpor to early arousal transition (25 cases), followed by late arousal to early torpor comparison (this is not a transition in the hibernation sense but rather a comparison of two states) (11 cases), early to late arousal transition (10 cases), and early to late torpor transition (6 cases). The expression of four significant genes in four-stage analysis in real-time PCR including Adfp in BAT, Atf4 in liver, Cact in heart, and Cyp51a1 in hypothalamus is shown in FIG. 2(A-D) together with the expression of these genes as measured in beadarray experiments.

### Example 3

### Significance of Hibernation-Related Genes

All statements of differential gene expression in this section are based on real-time PCR data.

Brown Adipose Tissue

In mammalian hibernators, BAT is essential for heat production through NST during hibernation. Our previous study on BAT in AGS with mouse microarrays (Yan et al. 2006) showed that the genes involved in the NST pathway are significantly up-regulated, whereas the ribosomal protein genes are significantly down-regulated in winter torpid animals compared to summer active animals. In this study, fatty acid catabolic genes (Hsl, Cpt1a, Cpt1b, Acadm, Acadvl, Hadha, and Cact) and TCA cycle genes (Idh2 and Mdh2) are significantly (P<0.05) up-regulated and uncoupling protein 1 (Ucp1) was moderately (P=0.08) up-regulated whereas ribosomal protein S16 (Rps16) was significantly down-regulated in torpid animals compared to NHC, which is completely consistent with our previous study.

As we included aroused animals in this study, we further investigated variation in the expression of these genes throughout the torpor-arousal cycle. Among these genes, we observed that Cpt1a, Acadm, Acadvl, Hadha, Cact, Idh2, Mdh2, and Rps16 remain unchanged (P>0.1) whereas Cpt1b and Hsl are significantly down-regulated (P<0.05) and Gpd1, Bckdhb, and Cs are moderately down-regulated (0.05<P<0.1) in aroused animals compared to torpid animals using the three-stage analysis. The down-regulation is most significant as AGS enter early arousal from late torpor using the four-stage analysis. As these genes are maintained at a high level in torpor, a drop of their mRNA levels in arousal may be a result of the high metabolism and thermogenesis as animals rewarm from torpor that deplete their mRNA transcripts through rapid translation and subsequent degradation. This could be a general phenomenon, as down-regulation in aroused animals compared to torpid animals was also observed for some other metabolic genes in liver (Acaa1 and Cox5b) and heart (Pdk2 and Cpt1b).

Phosphoenolpyruvate carboxykinase (Pck1 and Pck2) are key enzymes in gluconeogenesis. In BAT, Pck1 was significantly (P<0.01) and Pck2 was moderately (P=0.09) up-regulated in BAT. In addition, Pck1 was significantly down-regulated in aroused animals compared to torpid animals. This perhaps can also be explained by the above-mentioned depletion of mRNA during torpor-arousal transition. Although liver has been considered the major organ for gluconeogenesis, our results show that BAT can also contribute to the up-regulation of glucose synthesis during hibernation.

The substrate of gluconeogenesis in BAT is most likely glycerol, as hormone-sensitive lipase (Hsl) cleaves triglyceride into free fatty acid and glycerol. Fatty acids fuel NST in BAT. Glycerol is phosphorylated by glycerol kinase into glycerol 3-phosphate which is subsequently oxidized by glycerol-3-phosphate dehydrogenase into dihydroxyacetone phosphate which enters gluconeogensis. In support of this, glycerol-3-phosphate dehydrogenase 1 (Gpd1) had a similar expression profile as Hsl.

Antioxidant enzymes: selenoprotein P (Sepp1) and peroxiredoxin 6 (Prdx6) were significantly down-regulated in both torpid and aroused animals compared to NHC. Sepp1 is the only selenoprotein known to contain more than one selenocysteines and is also involved in selenium transport (Hill et al. 1993). Its down-regulation may indicate that selenium transport is inactive during hibernation.

Pyruvate dehydrogenase kinase, isozyme 4 (Pdk4) was significantly up-regulated and its isoform: Pdk1 was also moderately (P=0.1) up-regulated in BAT in both aroused and torpid animals compared to NHC. Pyruvate dehydrogenase kinase inactivates pyruvate dehydrogenase by phosphorylation and, therefore, blocks the conversion of pyruvate to acetyl-CoA in carbohydrate catabolism. Pdk4 has previously been shown to be up-regulated during torpor in heart, skeletal muscle, and white adipose tissue of thirteen-line ground squirrels supporting that carbohydrate metabolism is shifted to fatty acid metabolism during hibernation (Andrews et al. 1998, Buck et al. 2002).

In BAT, NST is activated by adrenergic stimulation through the β₃-adrenoceptor. Adenylate cyclase interacts with Gₛ protein which is linked to β₃-adrenoceptor. Adenylate cyclase activates the rise of second messenger cAMP, initiating further downstream signaling events. Four isoforms of adenylate cyclase: Acdy3, Acdy4, Acdy6, and Acdy9 are expressed in BAT. Only Acdy3 has been previously shown to be up-regulated as the result of increased adrenergic simulation during BAT differentiation (Chaudhry and Granneman 1997, Chaudhry et al. 1996). Here the up-regulation of adenylate cyclase 6 (Adcy6) in both aroused and torpid animals compared to NHC indicates the enhanced adrenergic simulation of NST in BAT during hibernation. The down-regulation of insulin-like growth factor 2 (Igf2) during both torpor and arousal in BAT is consistent with the finding of Schmidt and Kelley (2001) showing that insulin-like growth factor 1 (Igf1) has 75% reduction in the serum of golden-mantle ground squirrels during hibernation, suggesting insulin-like growth factor regulation of somatic growth is suppressed during hibernation as part of the energy-saving strategy.

Liver

Liver is where we observed the most significant differential expression in diverse functional categories. In the absence of food ingestion, glucose becomes limited during hibernation. Gluconeogenesis in liver can provide glucose to organs like the brain, where glucose is the major energy source. In liver, genes involved in gluconeogenesis (Pck1, Pck2, and G6pd) are significantly up-regulated in both aroused and torpid animals compared to NHC. In contrast, a key enzyme in glycolysis, hexokinase 4 (Hk4) or glucokinase, was significantly down-regulated by as much as 32-fold in both aroused and torpid animals compared to NHC. Pyruvate dehydrogenase beta (Pdhb) was slightly up-regulated whereas Pyruvate dehydrogenase kinases (Pdk1 and Pdk4) are not significantly changed in both aroused and torpid animals compared to NHC.

Glycogen is an important energy store in liver. In glycogen synthesis, glucose-1-phosphate is converted to UDP-glucose by UDP-glucose pyrophosphorylase (Ugp2) and subsequently converted to glycogen by glycogen synthase (Gys1 and Gys2), where Gys1 is muscle specific and Gys2 is liver specific. Both Ugp2 and Gys2 were significantly down-regulated whereas Gys1 was not significantly changed in both aroused and torpid animals compared to NHC. The down-regulation of Ugp2 and Gys2 are consistent with the above-mentioned down-regulation of Hk4 since Hk4 is responsible for converting glucose-6-phosphate to glucose-1-phosphate in glycolysis, which also acts as the first step of glycogen synthesis.

On the other hand, glycogen break-down is catalyzed by glycogen phosphorylase (Pygb). Pygb was significantly up-regulated in both aroused and torpid animals compared to NHC. These observations suggest that glycogen synthesis is suppressed and glycogen break-down is favored in both aroused and torpid animals compared to NHC. It has been hypothesized that glycogen storage is depleted during torpor and replenished during arousal (Galster and Morrison 1975). Gsy2 does show significant up-regulation in aroused animals compared to torpid animals. However, glycogen synthase kinase (Gsk3a and Gsk3b), which phosphorylates and inactivates glycogen synthase, is also significantly up-regulated in aroused animals compared to torpid animals. It is possible that increased glycogen synthase may still remain in the largely inactive form and glycogen synthesis remains suppressed during arousal.

Fatty acid β-oxidation (Fabp1, Acaa1, Acaa2, Acadvl, Hadhsc, and Cpt1a) was significantly up-regulated in both torpid and aroused animals compared to NHC. This is consistent with the paradigm that carbohydrate catabolism is shifted to fatty acid catabolism during hibernation. However, unlike Acaa1 and Acaa2, Acat2 was significantly down-regulated in both aroused and torpid animals compared to NHC. This may be related to the role of Acat2 in cholesterol metabolism, as another gene in cholesterol biosynthesis, Sc4mol, was also down-regulated. Contrary to fatty acid catabolism, fatty acid biosynthesis (Scd, Acacb, Elovl6, Sc4 mol, and Agpat3) was significantly down-regulated in aroused and torpid animals compared to NHC.

Among the genes involved in amino acid metabolism, As, Cps1, and Arg1 in urea cycle and Pah and Hpd in phenylalanine catabolism were significantly down-regulated whereas Glud1, Got1, and Got2 were significantly up-regulated in both torpid and aroused animals compared to NHC. Got1 and Got2 are two isozymes of aspartate aminotransferase. Aminotransferases and glutamate dehydrogenase together convert amino acid into α-ketoglutarate, which can enter the TCA cycle and gluconeogenesis. This may indicate a redirection of amino acid from urea cycle to gluconeogenesis and TCA cycle. In fact, the up-regulation of gluconeogenesis enzymes together with aminotransferases and glutamate dehydrogenase has already been observed in caloric restricted mouse liver (Hagopian et al. 2003). Galster and Morrison (1975) showed that glucose is replenished during arousal presumably through gluconeogenesis in AGS, with three-fourths estimated from fat and one-fourth from protein. Whitten and Klain (1968) showed that protein catabolism is increased during arousal in thirteen-line ground squirrels. However, our results show that there is no significant variation of mRNA levels of the genes involved in either gluconeogenesis or amino acid metabolism in liver during the torpor-arousal cycles.

A large number of transporters are significantly up-regulated in both torpid and aroused animals compared to NHC. These include: Alb in steroid, fatty acid, and thyroid hormone transport; Slc16a12 or Mct12 in lactate, pyruvate, and ketone body transport; Laptm4a in small molecule transport; Trappc5 in vesicle mediated transport; Abcb7 in heme transport; Col18a1 in phosphate transport; Tf in ferric ion transport. This indicates that liver is speeding up transport to distribute various “cargos” more efficiently in response to the limited supplies during hibernation.

We also observed that genes involved in xenobiotic metabolism or detoxication are significantly down-regulated in both torpor and arousal compared to NHC. These include: cytochrome P450 (Cyp1a2 and Cyp51a1) and flavin-containing monooxygenase (Fmo5) in drug, cholesterol, and steroid metabolism, UDP-glucuronosyltransferase (Ugt1a9) in steroids, bilirubin, hormones, and drug metabolism, and carboxylesterase 1 (Ces1) in cocaine and heroin metabolism. Three genes with anti-oxidant activities (Cat, Prdx6, and Mgst1) were significantly down-regulated in both aroused and torpid animals compared to NHC. This suggests that pathways that are energetically costly but not crucial to survival are actively suppressed as part of the energy-saving strategy of hibernation.

Alpha-2-macroglobulin (A2m), amyloid P component, serum (Apcs), and inter-alpha (globulin) inhibitor H4 (Itih4) involved in acute phase response were all significantly up-regulated in both aroused and torpid animals compared to NHC. The up-regulation of A2m during hibernation is well-established and has been suggested to increase the blood clotting time during hibernation in various hibernating species. Srere et al. (Srere et al. 1995) showed that, unlike A2m, several other acute phase proteins including Apcs were not significantly changed in hibernating Richardson's ground squirrels compared to active animals. Therefore, they suggested that the up-regulation of A2m is independent of acute phase response. Our observation of the up-regulation of Apcs in hibernating AGS is perhaps due to the species difference and again raises the possibility of activation of acute phase response during hibernation.

Heart

The heart of arctic ground squirrels can maintain contractile function in torpor, as the heart rate decreases to 1% of the euthermic level and tissue temperature to near 0° C., whereas non-hibernating mammals develop cardiac arrhythmias and ventricular fibrillation under hypothermia. In heart, we find that myosin light polypeptide 6 (Myl6) was significantly up-regulated in both torpid and aroused animals compared to NHC. The change of composition of myosin isoforms has been implicated in enhancing the contractility of hibernating heart (Morano 1999, Morano et al. 1992, Morano et al. 1995). Brauch et al. (2005) found the down-regulation of myosin light polypeptide 3, ventricular isoform (Myl3) and up-regulation of myosin heavy polypeptide 6, cardiac muscle, alpha (Myh6) in the heart of thirteen-lined ground squirrels in torpor compared with summer active animals, whereas Fahlman et al. found Myl3 is up-regulated in hibernating golden-mantle ground squirrels (Fahlman et al. 2000).

Maintenance of intracellular Ca²⁺ homeostasis is also important for contractile function of heart at low temperature (Liu et al. 1997, Wang et al. 2002). Sarco(endo)plasmic reticulum Ca²⁺-ATPase 2a (Serca2a or Atp2a2), a Ca²⁺ pump located on the sarcoplasmic/endoplasmic reticulum (SR/ER) membrane responsible for Ca²⁺ removal from cytosol, has been shown to be up-regulated during torpor in several hibernating species (Brauch et al. 2005, Yatani et al. 2004). This partially explained the enhanced cytoplasmic Ca²⁺ clearance and larger Ca²⁺ store in SR. In our study, Atp2a2 was moderately up-regulated (P=0.09) in torpid animals compared to NHC and significantly (P=0.05) down-regulated in aroused animals compared to torpid animals. In addition, ryanodine receptor 2 (Ryr2), a Ca²⁺ release channel on SR membrane, was significantly down-regulated in aroused animals compared to torpid animals.

Transmembrane emp24 protein transport domain containing 4 (Tmed4) or glycoprotein 25L (GP25L) was significantly up-regulated in both torpid and aroused animals compared to NHC. GP25L has been shown to be a member of transmembrane protein complexes on endoplasmic reticulum (ER) with Ca²⁺ binding capability (Wada et al. 1991). This may further contribute to the enhanced Ca²⁺ clearance from cytosol, to avoid Ca²⁺ overload in the hibernating heart.

Fatty acid catabolic genes (Cpt1a, Cpt1b, and Cact/Slc25a20) were significantly up-regulated in torpid animals compared to NHC and moderately down-regulated in aroused animals compared to torpid animals. Fatty acid binding proteins are responsible for transporting free fatty acids. Heart type fatty acid binding protein (Fabp3) was significantly up-regulated in torpid and aroused animals compared to NHC whereas adipose type fatty acid binding protein (Fabp4) was significantly down-regulated in aroused animals compared to torpid animals in heart.

Pyruvate dehydrogenase beta (Pdhb) was significantly up-regulated in torpid and aroused animals compared to NHC. However, similar to the situation in BAT, Pdk4 was significantly up-regulated thus inactivates pyruvate dehydrogenase by phosphorylation. Pdk2 was also significantly up-regulated in torpid animals compared to NHC but significantly down-regulated in aroused animals compared to torpid animals.

Uncoupling protein 2 (Ucp2) was significantly up-regulated in torpid and aroused animals compared to NHC in heart. Probes for all three homologs of uncoupling proteins, Ucp1, Ucp2, and Ucp3, all exist on our beadarrays. Ucp1 was exclusively detected and moderately up-regulated in torpid and aroused animals compared to NHC in BAT. Ucp2 was detected and significantly up-regulated in both BAT and heart, whereas Ucp3 was not detected in any tissue. Although Ucp2 and Ucp3 are unlikely to be involved in NST, their functions are still unclear (Cannon and Nedergaard, 2004). Ucp2 was expressed in multiple tissues including WAT, spleen, and heart, whereas Ucp3 was expressed mainly in skeletal muscle. It also has been shown that Ucp2 is up-regulated in WAT and Ucp3 in skeletal muscle in hibernating arctic ground squirrels (Boyer et al. 1998). The up-regulation of Ucp2 has been suggested to be part of an antioxidant defense response in the heart under oxidative stress (Teshima et al. 2003) and/or ischemia (McLeod et al. 2005). The up-regulation of Ucp2 in both BAT and heart observed herein supports the conclusion that it has a more general role in hibernation.

Hypothalamus

The hypothalamus plays an important role in regulating thermogenesis, metabolic rate, feeding, and circadian rhythms. It maintains body homeostasis by directing compensatory changes through autonomic, endocrine, and behavioral responses. The suprachiasmatic nucleus (SCN) that contains the master circadian clock is also located in hypothalamus. The hypothalamus senses body temperature and defends the temperature set-point by regulating thermogenesis.

During entry into torpor, the temperature set-point is gradually lowered from euthermic temperature ˜37° C. to near zero degrees (Heller et al. 1977). Hypothalamic controls of thermogenesis and, potentially, circadian rhythm still persist even in the absence of action potentials, as brain temperature decreases below 15° C. (Miller et al. 1994, Krilowicz et al. 1988, Heller and Ruby, 2004). The hypothalamus may play a key role in the entrance to and arousal from torpor during the torpor-arousal cycles (Kilduff et al. 1990). Despite the importance in hibernation, very few molecular studies have been carried out on hypothalamus during hibernation. So far, only c-fos, junB, and c-Jun of the immediate early genes have been shown to be slightly up-regulated in the hypothalamus during torpor and significantly up-regulated during arousal, and PGD2 synthase was found to decline during late torpor but to return to its former level during arousal (Bitting et al. 1994, O'Hara et al. 1999).

Early glucose uptake labeling studies in the brains of hibernating ground squirrels showed that hypothalamic regions are activated whereas cortical regions are inhibited relative to other regions of the brain during the entrance into hibernation (Kilduff et al. 1990). Among all glycolytic enzymes tested in our study, only Hk1 was significantly up-regulated in aroused animals compared to NHC whereas Gapd, Pfkm, and Pkm2 remain unchanged. Among the fatty acid catabolic enzymes, Acs13 and Acadm were significantly up-regulated whereas Acaa2 was significantly down-regulated in torpid animals compared to NHC.

In addition, Mdh2 involved in the TCA cycle and Cox5b involved in electron transport were up-regulated in torpid animals compared to NHC. Mdh2 was further up-regulated whereas Cox5b was down-regulated in aroused animals compared to torpid animals.

Among the genes involved in amino acid metabolism, Bckdhb was significantly up-regulated whereas Glud1 was significantly down-regulated in both torpid and aroused animals compared to NHC. Glud1 is responsible for converting glutamate, an important excitatory neurotransmitter, into α-ketoglutarate. The down-regulation of Glud1 may lead to an increase of glutamate level in hypothalamus. 4-aminobutyrate aminotransferase (Abat) was significantly up-regulated in aroused animals compared to torpid animals and NHC. Abat is responsible for catabolism of gamma-aminobutyric acid (GABA), an important inhibitory neurotransmitter in the central nervous system. The up-regulation of Abat may lead to a drop of GABA level in hypothalamus during arousal.

GABA imbalance in the brain has been implicated in various neurological disorders. Lust et al. (1989) observed elevated GABA levels in the brain of hibernating hamsters and it was suggested to be a neuronal depression mechanism, whereas Osborne et al. (1999) showed that GABA is actually decreased in the striatum of hibernating arctic ground squirrel using quantitative microdialysis. In hypothalamus, the up-regulation of Abat can be particularly important for the control of NST in BAT during arousal in that the inhibitory signal from preoptic/anterior hypothalamus (POAH) to ventromedial nucleus (VMN) is mediated by GABA along the thermoregulatory pathway from hypothalamus to BAT (Cannon and Nedergaard, 2004).

Elongation of long chain fatty acid (Elovl6) and stearoyl-CoA desaturase (Scd) are involved in elongation and desaturation of long chain fatty acids and were significantly up-regulated in aroused animals compared to torpid animals. A member of cytochrome P450 family, Cyp51a1, involved in xenobiotic metabolism was also significantly up-regulated in aroused animals compared to torpid animals and NHC, which may indicate that metabolism of toxins is increased during arousal from torpor in hypothalamus. In addition, cytochrome P450 proteins as monooxygenases also participate in the oxidative reaction in fatty acid desaturation together with Scd. Therefore, its up-regulation in aroused animals is consistent with that of Scd. However, acetyl-Coenzyme A carboxylase beta (Acacb), responsible for converting acetyl-CoA to malonyl-CoA in the first step of fatty acid biosynthesis, was significantly down-regulated in aroused animals compared to torpid animals. Cyp51a1 is also involved in other types of metabolism more relevant than xenobiotic metabolism. A portion of the GeneCard entry for Cyp51a1 is provided below.

### Official Full Name

cytochrome P450, family 51, subfamily A, polypeptide 1

### Gene Type

protein coding

### Organism

Homo sapiens

### Summary

This gene encodes a member of the cytochrome P450 superfamily of enzymes. The cytochrome P450 proteins are monooxygenases which catalyze many reactions involved in drug metabolism and synthesis of cholesterol, steroids and other lipids. This endoplasmic reticulum protein participates in the synthesis of cholesterol by catalyzing the removal of the 14alpha-methyl group from lanosterol. Homologous genes are found in all three eukaryotic phyla, fungi, plants, and animals, suggesting that this is one of the oldest cytochrome P450 genes.

In contrast to hypothalamus, Elovl6, Scd, Cyp51a1, and Acacb were all down-regulated in both aroused and torpid animals compared to NHC in liver. Ras suppressor protein 1 (Rsu1) inhibits the tumor growth in breast cancer and glioma cell lines by suppressing Ras signal transduction pathway (Chunduru et al. 2002). Our results showed that Rsu1 was up-regulated in torpid animals compared to NHC but down-regulated in aroused animals compared to torpid animals. Rsu1 can increase the activation of extracellular signal-regulated kinase (Erk) pathway, one of mitogen-activated protein kinase (MAPK) signal transduction pathways. Elevated expression of Rsu1 enhances extracellular signal-regulated kinase 2 (Erk-2) activation and inhibits Jun kinase activation (Masuelli and Cutler. 1996).

Guanine nucleotide binding protein (G protein), alpha activating activity polypeptide O (Gnao1 or Goα1), one of the most abundant G proteins expressed in the brain, is also involved in the mediation of extracellular signal-regulated kinase (Erk) activation through delta opioid receptor in neural cells (Zhang et al. 2003). Like Rsu1, Gnao1 was also up-regulated in torpid animals compared to NHC but down-regulated in aroused animals compared to torpid animals. The differential expression of Gnao1 together with Rsu1 during hibernation suggests variation in the Erk pathway in hypothalamus. In fact, several studies have already pointed out the differential regulation of Erk activation during hibernation in various species.

Zhu et al. (2005) showed that Erk is activated in the brain of both aroused and euthermic non-hibernating AGS compared to torpid AGS. They suggested that the activation of the Erk pathway is associated with the elevated hypoxia inducing factor 1 alpha (Hif1a) level as a neuroprotective mechanism against ischemia and/or reperfusion in the brain during arousal. However, there are also reports that the activation of Erk is increased during torpor in the brain of Richardson's ground squirrels (S. richardsonii) compared to euthermic non-hibernating ground squirrels (MacDonald and Storey, 2005) and decreased during arousal in the brain of bat (Rhinolopus ferrumequinum) compared with bat in torpor (Lee et al. 2002). Erk is implicated in the regulation of the circadian clock located in the SCN of hypothalamus. The levels of active and phosphorylated forms of ERK exhibit circadian variation in the SCN, with high levels during the subjective day and low levels during the subjective night and can also be rapidly induced by light pulses during the subjective night (Serchov and Heumann, 2006).

Proteasome is involved in the proteasome-dependent degradation of clock genes in generating the circadian oscillation. A subunit of proteasome (Psma7) was significantly up-regulated in aroused and torpid animals compared to NHC. The mRNA levels of proteasome components and Ras/MAPK signaling genes have already been shown to undergo circadian oscillation together with canonical clock genes in an in vitro circadian system in rat (Duffield et al. 2002). Basic helix-loop-helix transcription factor Bhlhb2 or Dec1, a member of the fifth clock gene family, was significantly up-regulated in torpid animals compared to NHC and further up-regulated in aroused animals. In mouse, Dec1 and Dec2 repress Clock/Bmal1-induced activation of Per1 and are expressed in SCN in a circadian fashion, with a peak in the subjective day (Honma et al. 2002). It has been suggested that the circadian clock in the SCN plays a key role in regulating the timing of torpor-arousal bout during hibernation season (Heller and Ruby, 2004). Our results suggest that the genes involved in circadian rhythm may undergo oscillation during torpor-arousal cycle. It may be that the molecular circuit generating circadian rhythm in the SCN is rewired to drive torpor-arousal cycle during hibernation.

Skeletal Muscle

Skeletal muscle is only transiently active during the early phase of arousal from torpor through intensive shivering thermogenesis and remains inactive during the multi-day torpor bout. Despite the extended period of inactivity during hibernation, the effect of disuse atrophy is significantly reduced compared with non-hibernating species (Steffen et al. 1991, Wickler et al. 1991). Two key enzymes involved in glycolysis (Pfkm and Pkm2) were significantly down-regulated in both torpid and aroused animals compared to NHC, whereas Pdk4 was significantly up-regulated in muscle. The down-regulation of Pfkm is consistent with a previous study that showed the enzyme activity of Pfkm is decreased during torpor in skeletal muscle, indicating suppressed glycolysis (Macdonald and Storey, 2005b).

Among fatty acid catabolic genes, Cpt1a, Cpt1b, and Hadhsc were significantly up-regulated whereas Gpd1 was significantly down-regulated in both torpid and aroused animals compared to NHC. Gpd1 is also involved in gluconeogenesis but the expression of a key enzyme in gluconeogenesis, Pck1, was not significantly changed in skeletal muscle.

Activating transcription factor 4 (Atf4) was down-regulated in both torpid and aroused animals compared to NHC. As a member of ATF/CREB family, Atf4 interacts with RNA polymerase II subunit 3 (RPB3) and positively regulates transcription in muscle (De Angelis et al. 2003). Atf4 is also up-regulated upon endoplasmic reticulum (ER) stress and/or hypoxia and activates the genes involved in amino acid metabolism and anti-oxidant defense (Harding et al. 2003).

Carbonic anhydrase III (Ca3) and creatine kinase muscle (Ckm) were down-regulated in both torpid and aroused animals compared to NHC. Ca3 has anti-oxidant function (Zimmerman et al. 2004) and therefore its down-regulation is consistent with the down-regulation of Atf4. The mRNA levels of Ca3 and Ckm are reduced in the skeletal muscle during fasting in mouse (Jagoe et al. 2002), responding to muscle atrophy. Furthermore, serum levels of both Ca3 and Ckm proteins are significantly increased in human patients with muscle dystrophy, especially Duchenne muscle dystrophy, which is most likely due to the loss of these proteins in skeletal muscle (Mokuno et al. 1987). Jagoe et al. (2002) showed that mRNA levels of Ca3 and Ckm together with some glycolytic enzymes are significantly reduced whereas that of Pdk4 is significantly increased in the skeletal muscle of fasting mouse. Our results are consistent with their findings, indicating that skeletal muscles in hibernating arctic ground squirrels undergo similar gene expression changes to atrophying muscles. Ribosomal protein Rps2, involved in protein biosynthesis, was significantly up-regulated in torpid and aroused animals compared to NHC whereas Lgmn, involved in proteolysis, was significantly down-regulated, which may play an important role in preserving the protein content in skeletal muscle during hibernation.

Differential Gene Expressions Shared Among Tissues

The differential expression of many genes during hibernation is tissue-specific related to the different functions of organs and tissues. There are also many gene expression patterns shared across the different tissues, reflecting the common challenges faced by all tissues during hibernation. The most prominent common response shared among the tissues is the metabolic shift from carbohydrate catabolism to fatty acid catabolism. This is manifested by down-regulation of glycolytic enzymes, up-regulation of fatty acid catabolic enzymes, and up-regulation of pyruvate dehydrogenase kinases in all tissues. Even for the hypothalamus, which is considered to rely mostly on glycolysis, the up-regulation of fatty acid catabolic enzymes like Acs13 and Acadm was observed. The up-regulation of Pdk4 in both torpid and aroused animals compared to NHC was observed in BAT, heart, and skeletal muscle. The up-regulation of gluconeogenesis was observed in both BAT and liver.

RNA binding motif protein 3 (Rbm3) was previously shown to be up-regulated during torpor in liver, heart, and brain of golden-mantle ground squirrels (Williams et al. 2005). Here we showed that Rbm3 was up-regulated in torpid and aroused animals compared to NHC in all tissues that we studied. As RNA binding proteins may have general functions such as RNA protection or translation inhibition, their up-regulation is consistent with the observation that mRNA transcripts are protected during torpor from degradation (Knight et al. 2000). In further support of this, RNase inhibitor H (Rnh1), with RNA protection function, was also significantly up-regulated in torpid and aroused animals compared to NHC in heart and skeletal muscle and up-regulated in aroused animals compared to torpid animals and NHC in hypothalamus.

Adipocyte differentiation-related protein or adipophilin (Adfp) was up-regulated in torpid and aroused animals compared to NHC in BAT, heart, skeletal muscle and up-regulated in aroused animals compared to torpid animals and NHC in liver and hypothalamus. Adfp was originally found to be up-regulated in BAT of torpid animals compared to summer active animals in our previous study and proposed to enhance the thermogenic capacity in BAT (Yan et al. 2006). In light of its ubiquitous up-regulation common to all tissues, Adfp may have more general functions, such as enhancing fatty acid metabolism.

CGI-69 protein was significantly up-regulated in torpid and aroused animals compared to NHC in BAT, liver, and heart. Yu et al. (2001) previously showed that CGI-69 is a mitochondrial carrier and is up-regulated 2-fold upon cold exposure in the BAT of mice. They further proposed CGI-69 to be another homolog of uncoupling proteins, but transfection of CGI-69 failed to change mitochondrial membrane potential, therefore casting doubt on its uncoupling activity.

The up-regulation of transporter genes and down-regulation of xenobiotic and anti-oxidant genes are also likely shared by several tissues. For example, the up-regulation of Tf and Laptm4a involved in transport was also found in both liver and hypothalamus. The down-regulation of Fmo5 in xenobiotic metabolism and Prdx6 in anti-oxidant metabolism was also found in both BAT and liver.

Wsb2 was up-regulated in torpid and aroused animals compared to NHC in heart and skeletal muscle. The functional significance of Wsb2 in hibernation is still unclear. Hemoglobin, alpha 1 (Hba1) was significantly down-regulated in heart and hypothalamus and moderately in BAT in torpid and aroused animals compared to NHC, which may reflect the reduced need of oxygen transport in tissues as a result of suppressed metabolism during hibernation.

Two heat shock proteins, Hspe1 and Hsp90ab1, were shown to be down-regulated in torpid AGS compared to summer active AGS in our previous results in BAT (Yan et al. 2006). In the present study, Hsp90ab1 was significantly down-regulated in heart and skeletal muscle in torpid animals compared to NHC. However, Hpse1 was significantly down-regulated in liver but up-regulated in heart in torpid animals compared to NHC. In addition, Hsp90ab1 was significantly up-regulated in liver and skeletal muscle in aroused animals compared to torpid animals, whereas no significant difference between aroused and torpid animals was found in Hspe1 in any tissue.

The up-regulation of Hsp90ab1 during arousal may help to maintain the proper folding of the proteins when the body temperature undergoes dramatic rise to euthermic level during early phase of arousal. It is still unclear why Hsp90ab1 and Hspe1 are down-regulated in certain tissues when the body temperature is near 0° C. during torpor. The different expression patterns between Hsp90ab1 and Hspe1 during hibernation may indicate the subtle functional differences between these two heat shock proteins.

Hist1h2a1 was found to be significantly up-regulated in torpid animals compared to NHC and down-regulated in aroused animals compared to torpid animals in both heart and hypothalamus. As a subunit of histone, the up-regulation of Hist1h2a1 during torpor may lead to a closed state of histone-DNA complex, therefore switching off general transcription. Furthermore, the down-regulation of Hist1h2a1 during arousal in heart and hypothalamus may lead to the re-activation of transcription, consistent with the up-regulation of transcription factors c-myc and Atf4 during arousal in liver.

### Modulation in Torpor-Arousal Cycles

A leading theory explaining the function of periodic arousal during hibernation proposes that gene products, i.e. mRNA transcripts and proteins, slowly degrade during torpor but are replenished during arousal. However, the above results showed that the mRNA levels of most genes are very stable during torpor-arousal cycles. In fact, out of 303 cases tested in Real-time PCR, only 61 cases (˜20%) show significant variations during torpor-arousal cycle. Although it has been shown that transcription is suppressed at low body temperature, the up-regulation of RNA binding protein and RNase inhibitor that protect the mRNA transcripts from degradation may explain the stability of the mRNA levels for most of the genes during torpor-arousal cycle.

Modulation of mRNA transcripts during torpor-arousal cycles does happen for a small portion of genes but not in a simple manner. Both up- and down-regulated genes were found in aroused animals compared to torpid animals in various tissues. The fact that the most significant differences in gene expression during torpor-arousal cycle happens in late torpor to early arousal transition, followed by late arousal to early torpor, is consistent with the dramatic physiological changes observed during early phase of arousal from torpor compared to the gradual re-entry into torpor from late arousal. In particular, among the 19 genes that were down-regulated comparing early aroused to late torpid animals, 13 of them are metabolic genes involved in fatty acid metabolism, amino acid metabolism, gluconeogenesis, and electron transport. This again may be explained by the high metabolism that is required during early arousal as animals rewarm from late torpor, depleting their mRNA transcripts through rapid translation and subsequent degradation.

Among the 6 genes that were up-regulated comparing early aroused animals to late torpid animals, c-myc, Atf4, Gsk3a, and Gsk3b in liver are implicated in cell growth and proliferation. c-myc is a key transcription factor regulating cell cycle progression, apoptosis, and cellular transformation. Its up-regulation is consistent with the previous observation that the immediate early genes including c-fos and c-jun were up-regulated during arousal in brain and other tissues in golden-mantled ground squirrel (O'Hara et al. 1999). Atf4 can form hetero-dimers with c-fos and c-jun (Hai and Curran, 1991). Atf4 also positively controls many genes involved in amino acid metabolism, transport, and anti-oxidant metabolism (Rutkowski and Kaufman, 2004). Potentially, Atf4 may also belong to the immediate early genes that promote cell growth and proliferation.

The down-regulation of Hist1h2a1 during arousal may lead to the dissociation of histones from DNA and make DNA accessible for transcription factors such as c-myc and Atf4 initiating a cascade of downstream transcriptional events. Gsk3a and Gsk3b are also implicated in cell cycle and proliferation in addition to their roles in glycogen synthesis. There is experimental evidence showing that the cell cycle is blocked at G₂ or late S phase during torpor but resumes during arousal in intestinal epithelial cells (Martin and Carey, 1996; Kruman et al. 1988). It was proposed that this can prevent the cells from possible damage in mitosis under hypothermia accompanying hibernation (Kruman et al. 1988).

Our results provide the first evidence of cell cycle arrest and resumption during the torpor-arousal cycle on the molecular level. The difference of gene expression between early torpor and late torpor is small for most of the genes examined. The mRNA level drops significantly only in 4 cases as animals proceed from early torpor to late torpor, whereas it increases in 2 cases. Therefore, there is no significant decay of the static mRNA level during torpor on a global scale.

Beadarray and Real-Time PCR Comparison

The fact that we had a high degree of agreement between beadarray and real-time PCR comparing torpid animals to NHC, but less agreement comparing aroused animals to torpid animals, reflects that the difference in gene expression between torpor and arousal is small relative to that between torpor and NHC. This probably explains why Williams et al. (2005) failed to detect any significant differences between aroused animals and torpid animals. The bias introduced at different steps in beadarray experiments, including sample labeling, hybridization, and normalization, can easily skew the magnitude or even the direction of differences in gene expression when the real difference is small, but will not have any significant effect when the real difference is large.

Most of the probes present on our beadarray were designed from the sequences of S. lateralis. Previous studies showed that about an average of 1% difference exists between S. lateralis and AGS mRNA sequences (Yan et al. 2006). On our beadarrays, one or two mismatches between the probes and labeled transcripts can considerably decrease the detection signals and low signals generally have larger fluctuation. For example, c-myc in liver was originally identified as significantly down-regulated in torpid animals compared to NHC on the beadarrays, while it was shown to be significantly up-regulated in aroused animals compared to both torpid animals and NHC in real-time PCR. The signals of c-myc in liver were very weak on beadarrays. In fact, the detection scores of c-myc were less than 0.99 in 16 out of 24 liver samples, which may contribute to its misclassification on beadarrays. Therefore, such discrepancy in sequences could contribute to the increased false positive and negative rates on beadarrays. On the other hand, although real-time PCR is much more sensitive than the hybridization-based assays, the fluctuations in sample preparations and the amounts of 18S gene in different samples can also diminish the significance of differences in real-time PCR when the real difference is small. Combining these two independent gene-profiling approaches is therefore important for the studies involving large sample sizes and multiple-state comparison.

### CONCLUSION

The results reported herein show the first systematic gene expression study on mammalian hibernation that includes multiple hibernation states and a wide range of tissues. The results support that the shift from carbohydrate to fatty acid catabolism is the major theme of gene expression reprogramming during hibernation, and shed new light on other aspects of metabolism like gluconeogenesis and amino acid metabolism in various tissues. Consistent with the common finding that the most discordant aspects of a phenotype are the most informative, between season differences in gene expression were more striking than within season differences. However, variation of gene expression during multiple stages of the torpor-arousal cycle does exist in a rather complex manner.

Our results provide considerable evidence in contrast to the traditional view that mammalian hibernators arouse to replenish mRNA transcripts. Instead, we observed a drop of expression during the transition from late torpor to early arousal for a group of metabolic genes. We propose that this is due to the exhaustion of mRNA transcripts during the energetic demands of the early arousal phase. We also observed a sharp rise of expression during late torpor to early arousal transition for the genes related to cell growth and proliferation. We propose that this reflects the resumption of cell cycle during arousal that has been stalled during torpor. Whether the similar modulation occurs at the protein level remains to be demonstrated. Based on our results in hypothalamus, we hypothesize that circadian clock genes may undergo differential expression during hibernation and therefore play an important role in regulating torpor-arousal cycles.

### Example 4

### Statistically Significant Hibernation-Related Genes

The expression levels of ten hibernation-related genes were found to show statistically significant (p<0.05) differences between torpid animals, active animals and/or NHC (non-hibernating control animals). As discussed above, Adfp (adipocyte differentiation-related protein or adipophilin) was up-regulated in torpid and arounsed animals compared to NHC in BAT, heart and skeletal muscle and was up-regulated in aroused animals compared to torpid animals and NHC in liver and hypothalamus. Adfp may function in enhancing fatty acid metabolism.

The Atf4 (activating transcription factor 4) gene encodes a transcription factor (also known as CREB-2) that mediates cAMP-dependent transcription. The protein binds to the cAMP response element (CRE) that is present in many viral and cellular promoters. Atf4 positively controls many genes involved in amino acid metabolism, transport, and anti-oxidant metabolism (Rutkowski and Kaufman, 2004). Potentially, Atf4 may also belong to the immediate early genes that promote cell growth and proliferation. Atf4 was down-regulated in both torpid and aroused animals compared to NHC. As a member of ATF/CREB family, Atf4 interacts with RNA polymerase II subunit 3 (RPB3) and positively regulates transcription in muscle (De Angelis et al. 2003). Atf4 is also up-regulated upon endoplasmic reticulum (ER) stress and/or hypoxia and activates the genes involved in amino acid metabolism and anti-oxidant defense (Harding et al. 2003). Atf4 expression was up-regulated in liver tissue during arousal.

Cact (carnitine/acycarnitine translocase) encodes a fatty acid catabolic protein that shuttles substrates in between the cytosol and mitochondrial matrix. Cact mRNA levels have been reported to be high in heart, skeletal muscle and liver (Huizing et al., 1998, J. Bioenerg. Biomemgr. 30:277-84). Cact expression was significantly up-regulated in torpid animals compared to NHC and moderately down-regulated in aroused animals compared to torpid animals. Cact showed significant modulation of expression during the torpor-arousal cycle in heart tissue.

Myosin light chain kinase 6 (Myl6) forms part of the contractile machinery in complex with myosin heavy chain. Myl6 has been reported to be expressed in smooth muscle and in non-muscle tissues (Lenz et al., 1989, J. Biol. Chem. 264:9009-15). Myl6 was significantly up-regulated in heart tissue in both torpid and aroused animals compared to NHC. Myl6 is involved in increased heart function during heart failure, although it is unknown whether it is present in smooth muscle or cardiac muscle in heart. Current histological studies are designed to examine the distribution of the Myl6 protein product in heart tissue. Distribution in cardiac blood vessels could indicate that Myl6 modulates blood pressure and/or cardiac blood flow during torpor/arousal cycles and may be of significance for cardiac function in various disease states. Myl6 exhibited the greatest change in gene expression levels of any hibernation-related gene examined to date, and exhibited over a 30-fold increase in expression in heart in torpor and arousal compared to NHC.

Carbonic anhydrase III (Ca3) catalyzes the reaction of CO₂ and water and is involved in pH regulation, ion transport and anti-oxidant function. Creatine kinase muscle (Ckm) catalyzes a transphosphorylation reaction between ATP and creatine, allowing short-term storage of high energy phosphodiester bonds. Both Ca3 and Ckm are found in high levels in skeletal muscle. Ca3 and Ckm were down-regulated in both torpid and aroused animals compared to NHC, associated with the down-regulation of Atf4. The mRNA levels of Ca3 and Ckm are reduced in the skeletal muscle during fasting in mouse (Jagoe et al. 2002), responding to muscle atrophy. Furthermore, serum levels of both Ca3 and Ckm proteins are significantly increased in human patients with muscle dystrophy, especially Duchenne muscle dystrophy, which is most likely due to the loss of these proteins in skeletal muscle (Mokuno et al. 1987). Jagoe et al. (2002) showed that mRNA levels of Ca3 and Ckm together with some glycolytic enzymes are significantly reduced whereas that of Pdk4 is significantly increased in the skeletal muscle of fasting mouse. Our results are consistent with their findings, indicating that skeletal muscles in hibernating arctic ground squirrels undergo similar gene expression changes to atrophying muscles.

Ribosomal protein S2 (Rps2) is involved in protein synthesis and has been suggested to function in binding of aminoacyl-tRNA to ribosomes and determining the fidelity of translation (Suzuki et al., 1991, J. Biol. Chem. 266:20007-10). Rps2 was significantly up-regulated in torpor and arousal, compared to NHC. Its role in protein synthesis suggests that Rps2 is important in the maintenance of muscle mass during inactivity, and may be significant in various forms of muscle wasting diseases.

Legumain (Lgmn) is a cysteine protease that is involved in proteolysis. In contrast to Rps2, Lgmn was significantly down-regulated in torpor and arousal compared to NHC and may play an important role in preserving skeletal muscle protein content during hibernation. The opposed roles of Rps2 and Lgmn in maintaining muscle mass may be of significance for dystrophies and other muscular diseases.

Cyp51a1 (lanosterol 14-alpha demethylase) encodes a member of the cytochrome P450 superfamily and is involved in drug detoxification and in cholesterol and steroid metabolism. Cyp51a1, involved in xenobiotic metabolism was also significantly up-regulated in the hypothalamus of aroused animals compared to torpid animals and NHC, which may indicate that metabolism of toxins is increased during arousal from torpor in hypothalamus. In addition, cytochrome P450 proteins as monooxygenases also participate in the oxidative reaction in fatty acid desaturation together with Scd. Therefore, its up-regulation in aroused animals is consistent with that of Scd. The GeneCard entry for Cyp51a1 discusses other metabolic functions for the protein. In contrast to hypothalamus, Cyp51a1 was down-regulated in both aroused and torpid animals compared to NHC in liver.

FABP (fatty acid binding protein) is involved in fatty acid β-oxidation and was significantly up-regulated in liver in both torpid and aroused animals compared to NHC. In heart tissue, heart type fatty acid binding protein (Fabp3) was significantly up-regulated in torpid and aroused animals compared to NHC whereas adipose type fatty acid binding protein (Fabp4) was significantly down-regulated in aroused animals compared to torpid animals.

The skilled artisan will realize that these ten hibernation-related genes, or their protein products, may be used as markers for detection or diagnosis of various disease states discussed above, or as targets for therapeutic treatment of such disease states. Probes, primers, detection moieties, inhibitors or activators of the genes and/or protein products may be used in the practice of the compositions or methods claimed herein.

All of the COMPOSITIONS and METHODS disclosed and claimed herein can be made and executed without undue experimentation in light of the present disclosure. While the compositions and methods have been described in terms of preferred embodiments, it is apparent to those of skill in the art that variations maybe applied to the COMPOSITIONS and METHODS and in the steps or in the sequence of steps of the methods described herein without departing from the concept, spirit and scope of the invention. More specifically, certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Altschul S F, Gish W, Miller W, Myers E W, and Lipman D J. Basic local alignment search tool. J Mol Biol 215: 403-410, 1990.
Andrews M T, Squire T L, Bowen C M, and Rollins M B. Low-temperature carbon utilization is regulated by novel gene activity in the heart of a hibernating mammal. Proc Natl Acad Sci USA 95: 8392-8397, 1998.
Barnes B M. Freeze avoidance in a mammal: body temperatures below 0 degree C. in an Arctic hibernator. Science 244: 1593-1595, 1989.
Bauer V W, Squire T L, Lowe M E, and Andrews M T. Expression of a chimeric retroviral-lipase mRNA confers enhanced lipolysis in a hibernating mammal. Am J Physiol Regul Integr Comp Physiol 281: R1186-R1192, 2001.
Bitting L, Sutin E L, Watson F L, Leard L E, O'Hara B F, Heller H C, and Kilduff T S. C-fos mRNA increases in the ground squirrel suprachiasmatic nucleus during arousal from hibernation. Neurosci Lett. 165: 117-121, 1994.
Boss O, Samec S, Dulloo A, Seydoux J, Muzzin P, and Giacobino J P. Tissue-dependent upregulation of rat uncoupling protein-2 expression in response to fasting or cold. FEBS Lett. 412: 111-114, 1997.
Boyer B B, Barnes B M, Kopecky J, Jacobsson A, and Hermanska J. Molecular control of prehibernation brown fat growth in arctic ground squirrels. In: Life in the Cold III: Ecological, Physiological and Molecular Mechanisms, edited by Carey C, Florant G L, Wunder B A, Horwitz B. Boulder (Colo.): Westview Press, 1993.
Boyer B B, Barnes B M, Lowell B B, and Grujic D. Differential regulation of uncoupling protein gene homologues in multiple tissues of hibernating ground squirrels. Am J Physiol Regul Integr Comp Physiol 275: R1232-R1238, 1998.
Boyer B B, and Barnes B M. Molecular and Metabolic Aspects of Mammalian Hibernation. BioScience 49: 713-724, 1999.
Bradshaw A D, Graves D C, Motamed K, and Sage E H. SPARC-null mice exhibit increased adiposity without significant differences in overall body weight. Proc Natl Acad Sci USA 100: 6045-6050, 2003.
Brauch K M, Dhruv N D, Hanse E A, and Andrews M T. Digital transcriptome analysis indicates adaptive mechanisms in the heart of a hibernating mammal. Physiol Genomics 23: 227-234, 2005.
Buck C L and Barnes B M. Effects of ambient temperature on metabolic rate, respiratory quotient, and torpor in an arctic hibernator. Am J Physiol Regul Integr Comp Physiol 279(1): R255-62, 2000.
Buck M J, Squire T L, and Andrews M T. Coordinate expression of the PDK4 gene: a means of regulating fuel selection in a hibernating mammal. Physiol Genomics 8: 5-13, 2002.
Buzadzic B, Spasic M, Saicic Z S, Radojicic R, Petrovic V M, and Halliwell B. Antioxidant defenses in the ground squirrel Citellus citellus. 2. The effect of hibernation. Free Radic Biol Med 9: 407-413, 1990.
Cannon B and Nedergaard J. Brown adipose tissue: function and physiological significance, Physiol Rev 84: 277-359, 2004.
Carey H V, Andrews M T, and Martin S L. Mammalian hibernation: cellular and molecular responses to depressed metabolism and low temperature. Physiol Rev 83: 1153-81, 2003.
Carey H V and Martin S L. Preservation of intestinal gene expression during hibernation. Am J Physiol Gastrointest Liver Physiol 271: G805-G813, 1996.
Chaudhry A and Granneman J G. Effect of hypothyroidism on adenylyl cyclase activity and subtype gene expression in brown adipose tissue. Am J Physiol Regul Integr Comp Physiol 273: R762-R767, 1997.
Chaudhry A, Muffler L A, Yao R, and Granneman J G. Perinatal expression of adenylyl cyclase subtypes in rat brown adipose tissue. Am J Physiol Regul Integr Comp Physiol 270: R755-R760, 1996.
Chunduru S, Kawami H, Gullick R, Monacci W J, Dougherty G, and Cutler M L. Identification of an alternatively spliced RNA for the Ras suppressor RSU-1 in human gliomas. J Neurooncol. 60: 201-211, 2002.
Daan S, Barnes B M, and Strijkstra A M. Warming up for sleep? Ground squirrels sleep during arousals from hibernation. Neurosci Lett. 128: 265-268 1991.
Daikoku T, Shinohara Y, Shima A, Yamazaki N, and Terada H. Dramatic enhancement of the specific expression of the heart-type fatty acid binding protein in rat brown adipose tissue by cold exposure. FEBS Lett 410: 383-386, 1997.
De Angelis R, Iezzi S, Bruno T, Corbi N, Di Padova M, Floridi A, Fanciulli M, and Passananti C. Functional interaction of the subunit 3 of RNA polymerase II (RPB3) with transcription factor-4 (ATF4). FEBS Lett. 547:15-19, 2003.
Duffield G E, Best J D, Meurers B H, Bittner A, Loros J J, and Dunlap J C. Circadian programs of transcriptional activation, signaling, and protein turnover revealed by microarray analysis of mammalian cells. Curr Biol. 12: 551-557, 2002.
Eddy S F, Morin P J, and Storey K B. Cloning and expression of PPAR-gamma and PGC-1 alpha from the hibernating ground squirrel, Spermophilus tridecemlineatus. Mol Cell Biochem 269(1-2): 175-182, 2005.
Eisen M B, Spellman P T, Brown P O, and Botstein D. Cluster analysis and display of genome-wide expression patterns. Proc Natl Acad Sci USA 95: 14863-14868, 1998.
Epperson L E, and Martin S L. Quantitative assessment of ground squirrel mRNA levels in multiple stages of hibernation. Physiol Genomics 10: 93-102, 2002.
Epperson L E, Dahl T A, and Martin S L. Quantitative Analysis of Liver Protein Expression During Hibernation in the Golden-mantled Ground Squirrel. Mol Cell Proteomics 3: 920-933, 2004.
Fahlman A, Storey J M, and Storey K B. Gene up-regulation in heart during mammalian hibernation. Cryobiology 40: 332-342, 2000.
Frerichs K U, Smith C B, Brenner M, DeGracia D J, Krause G S, Marrone L, Dever T E, and Hallenbeck J M. Suppression of protein synthesis in brain during hibernation involves inhibition of protein initiation and elongation. Proc Natl Acad Sci USA 95: 14511-14516, 1998.
Galster W and Morrison P R. Gluconeogenesis in arctic ground squirrels between periods of hibernation. Am J Physiol. 228: 325-330, 1975.
Gunderson K L, Kruglyak S, Graige M S, Garcia F, Kernani B G, Zhao C, Che D, Dickinson T, Wickham E, Bierle J, Doucet D, Milewski M, Yang R, Siegmund C, Haas J, Zhou L, Oliphant A, Fan J B, Barnard S, and Chee M S. Decoding Randomly Ordered DNA Arrays. Genome Res 14: 870-877, 2004.
Hai T and Curran T, Cross-family dimerization of transcription factors Fos/Jun and ATF/CREB alters DNA binding specificity. Proc Natl Acad Sci USA. 88:3720-3724, 1991.
Hagopian K, Ramsey J J, and Weindruch R. Caloric restriction increases gluconeogenic and transaminase enzyme activities in mouse liver. Exp Gerontol. 38:267-278, 2003.
Harding H P, Zhang Y, Zeng H, Novoa I, Lu P D, Calfon M, Sadri N, Yun C, Popko B, Paules R, Stojdl D F, Bell J C, Hettmann T, Leiden J M, and Ron D. An integrated stress response regulates amino acid metabolism and resistance to oxidative stress. Mol. Cell. 11: 619-633, 2003.
Heller H C, Colliver G W, and Bread J. Thermoregulation during entrance into hibernation. Pflugers Arch. 369: 55-59, 1977.
Heller H C and Ruby N F, Sleep and circadian rhythms in mammalian torpor. Annu Rev Physiol 66: 275-289, 2004.
Hill K E, Lloyd R S, and Burk R F. Conserved nucleotide sequences in the open reading frame and 3′ untranslated region of selenoprotein P mRNA. Proc Natl Acad Sci USA 90: 537-541, 1993.
Hittel D S and Storey K B. Differential expression of adipose- and heart-type fatty acid binding proteins in hibernating ground squirrels. Biochim Biophys Acta 1522: 238-243, 2001.
Hittel D S and Storey K B. Differential expression of mitochondria-encoded genes in a hibernating mammal. J Exp Biol 205: 1625-1631, 2002.
Honma S, Kawamoto T, Takagi Y, Fujimoto K, Sato F, Noshiro M, Kato Y, and Honma K. Dec1 and Dec2 are regulators of the mammalian molecular clock. Nature. 419: 841-844, 2002.
Jagoe R T, Lecker S H, Gomes M, and Goldberg A L. Patterns of gene expression in atrophying skeletal muscles: response to food deprivation. FASEB J. 16: 1697-1712, 2002.
Kilduff T S, Miller J D, Radeke C M, Sharp F R, and Heller H C. 14C-2-deoxyglucose uptake in the ground squirrel brain during entrance to and arousal from hibernation. J Neurosci. 10: 2463-2475, 1990.
Krilowicz B L, Glotzbach S F, and Heller H C. Neuronal activity during sleep and complete bouts of hibernation. Am J Physiol. 255: R1008-R1019, 1988.
Kruman I I, Ilyasova E N, Rudchenko S A, and Khurkhulu Z S. The intestinal epithelial cells of ground squirrel (Citellus undulatus) accumulate at G₂ phase of the cell cycle throughout a bout of hibernation. Comp Biochem Physiol A Physiol 90: 233-236, 1988.
Knight J E, Narus E N, Martin S L, Jacobson A, Barnes B M, and Boyer B B. mRNA stability and polysome loss in hibernating Arctic ground squirrels (Spermophilus parryii). Mol Cell Biol 20: 6374-6379, 2000.
Kuhn K, Baker S C, Chudin E, Lieu M H, Oeser S, Bennett H, Rigault P, Barker D, McDaniel T K, and Chee M S. A novel, high-performance random array platform for quantitative gene expression profiling. Genome Res. 14: 2347-2356 2004.
Lee M, Choi I, and Park K. Activation of stress signaling molecules in bat brain during arousal from hibernation. J Neurochem. 82: 867-873, 2002.
Liu B, Belke D D, and Wang L C. Ca2+ uptake by cardiac sarcoplasmic reticulum at low temperature in rat and ground squirrel. Am J Physiol Regul Integr Comp Physiol 272: R1121-R1127, 1997.
Long R A, T J Martin, and B M Barnes. Body temperature and activity patterns in free-living arctic ground squirrels. J Mammal 86(2): 312-322, 2005.
Lust W D, Wheaton A B, Feussner G, and Passonneau J, Metabolism in the hamster brain hibernation and arousal. Brain Res. 489: 12-20, 1989.
MacDonald J A and Storey K B. Mitogen-activated protein kinases and selected downstream targets display organ-specific responses in the hibernating ground squirrel. Int J Biochem Cell Biol. 37: 679-691, 2005.
MacDonald J A and Storey K B. Temperature and phosphate effects on allosteric phenomena of phosphofructokinase from a hibernating ground squirrel (Spermophilus lateralis). FEBS J. 272: 120-128, 2005.
Martin S, Srere H, Belke D, Wang L C H, and Carey H. Differential gene expression in the liver during hibernation in ground squirrels. In: Life in the Cold III: Ecological, Physiological and Molecular Mechanisms, edited by Carey C, Florant G L, Wunder B A, Horwitz B. Boulder (Colo.): Westview Press, 1993.
Martin S, Dahl T, and Epperson L E. Slow loss of protein integrity during torpor: a cause for arousal? In: Life in the Cold. Evolution, Adaptation, Mechanisms, and Applications, edited by Barnes B M and Carey H V. Fairbanks (Ak.): Institute of Arctic Biology, 2004.
Masuelli L and Cutler M L. Increased expression of the Ras suppressor Rsu-1 enhances Erk-2 activation and inhibits Jun kinase activation. Mol Cell Biol. 10: 5466-5476, 1996.
McLeod C J, Aziz A, Hoyt R F Jr, McCoy J P Jr, and Sack M N. Uncoupling proteins 2 and 3 function in concert to augment tolerance to cardiac ischemia. J Biol Chem. 280: 33470-33476, 2005.
Miller J D, Cao V H, and Heller H C. Thermal effects on neuronal activity in suprachiasmatic nuclei of hibernators and nonhibernators. Am J Physiol. 266: R1259-R1266, 1994.
Milner R E, Wang L C, and Trayhurn P. Brown fat thermogenesis during hibernation and arousal in Richardson's ground squirrel. Am J Physiol 256: R42-8, 1989.
Morano I. Tuning the human heart molecular motors by myosin light chains. J Mol Med 77: 544-555, 1999.
Morano I, Adler K, Agostini B, and Hasselbach W. Expression of myosin heavy and light chains and phosphorylation of the phosphorylatable myosin light chain in the heart ventricle of the European hamster during hibernation and in summer. J Muscle Res Cell Motil 13: 64-70, 1992.
Morano I, Ritter O, Bonz A, Timek T, Vahl C F, and Michel G. Myosin light chain-actin interaction regulates cardiac contractility. Circ Res 76: 720-725, 1995.
Mokuno K, Riku S, Sugimura K, Takahashi A, Kato K, and Osugi S. Serum creatine kinase isoenzymes in Duchenne muscular dystrophy determined by sensitive enzyme immunoassay methods. Muscle Nerve. 10: 459-463, 1987.
O'Hara B F, Watson F L, Srere H K, Kumar H, Wiler S W, Welch S K, Bitting L, Heller H C, and Kilduff T S. Gene expression in the brain across the hibernation cycle. J Neurosci. 19: 3781-3790, 1999.
Osborne P G, Hu Y, Covey D N, Barnes B M, Katz Z, and Drew K L. Determination of striatal extracellular gamma-aminobutyric acid in non-hibernating and hibernating arctic ground squirrels using quantitative microdialysis. Brain Res. 839: 1-6, 1999.
Pruitt K D, Tatusova, T, Maglott D R, NCBI Reference Sequence (RefSeq): a curated non-redundant sequence database of genomes, transcripts and proteins. Nucleic Acids Res 33:D501-D504, 2005.
Rutkowski D T and Kaufman R J, All Roads Lead to ATF4, Developmental Cell, 4:442-444, 2003.
Saitongdee P, Milner P, Becker D L, Knight G E and Burnstock G. Increased connexin43 gap junction protein in hamster cardiomyocytes during cold acclimatization and hibernation. Cardiovasc Res 47: 108-15, 2000.
Serchov T and Heumann R. Constitutive activation of ras in neurons: implications for the regulation of the Mammalian circadian clock. Chronobiol Int. 23: 191-200, 2006.
Schmidt K E and Kelley K M. Down-regulation in the insulin-like growth factor (IGF) axis during hibernation in the golden-mantled ground squirrel, Spermophilus lateralis: IGF-I and the IGF-binding proteins (IGFBPs). J Exp Zool 289: 66-73, 2001.
Smit A F, Hubley R and Green P. RepeatMasker Open-3.0. 1996-2004. (www.repeatmasker.org)
Soukri A, Valverde F, Hafid N, Elkebbaj M S, and Serrano A. Occurrence of a differential expression of the glyceraldehyde-3-phosphate dehydrogenase gene in muscle and liver from euthermic and induced hibernating jerboa (Jaculus orientalis). Gene 181: 139-145, 1996.
Srere H K, Wang L C H, and Martin S L. Central role for differential gene expression in mammalian hibernation. Proc Natl Acad Sci USA 89: 7119-7123, 1992.
Srere H K, Belke D, Wang L C H, and Martin S L. alpha 2-Macroglobulin gene expression during hibernation in ground squirrels is independent of acute phase response. Am J Physiol Regul Integr Comp Physiol. 268: 1507-1512, 1995.
Steffen J M, Koebel D A, Musacchia X J, and Milsom W K. Morphometric and metabolic indices of disuse in muscles of hibernating ground squirrels. Comp Biochem Physiol B. 99: 815-819, 1991.
Teshima Y, Akao M, Jones S P, and Marban E. Uncoupling protein-2 overexpression inhibits mitochondrial death pathway in cardiomyocytes. Circ Res. 93: 192-200, 2003.
Trachsel L, Edgar D M, and Heller H C. Are ground squirrels sleep deprived during hibernation? Am J Physiol Regul Integr Comp Physiol 260: R1123-R1129, 1991.
Tvrdik P, Kuzela S, and Houstek J. Low translational efficiency of the F1-ATPase beta-subunit mRNA largely accounts for the decreased ATPase content in brown adipose tissue mitochondria. FEBS Lett 313: 23-26, 1992.
Van Breukelen F and Martin S L. Translational initiation is uncoupled from elongation at 18C during mammalian hibernation. Am J Physiol Reg Comp Integrative Physiol 281: R1374-R1379, 2001.
Van Gelder R N, von Zastrow M E, Yool A, Dement A C, Barchas J D, and Eberwine E H. Amplified RNA synthesized from limited quantities of heterogeneous cDNA. Proc Natl Acad Sci USA 87: 1663-1667, 1990.
Wada I, Rindress D, Cameron P H, Ou W J, Doherty J J II, Louvard D, Bell A W, Dignard D, Thomas D Y, and Bergeron J J. SSR alpha and associated calnexin are major calcium binding proteins of the endoplasmic reticulum membrane. J Biol Chem 266: 19599-19610, 1991.
Wang S Q, Lakatta E G, Cheng H, and Zhou Z Q. Adaptive mechanisms of intracellular calcium homeostasis in mammalian hibernators. J Exp Biol 205: 2957-2962, 2002.
Whitten B K and Klain G, Protein metabolism in hepatic tissue of hibernating and arousing ground squirrel. Am J Physiol. 214: 1360-1362, 1968.
Wickler S J, Hoyt D F, and van Breukelen F. Disuse atrophy in the hibernating golden-mantled ground squirrel, Spermophilus lateralis. Am J Physiol. 261: R1214-1217, 1991.
Williams D R, Epperson L E, Li W, Hughes M A, Taylor R, Rogers J, Martin S L, Cossins A R, and Gracey A Y. Seasonally hibernating phenotype assessed through transcript screening. Physiol Genomics 24: 13-22, 2005.
Wilson B E, Deeb S, and Florant G L. Seasonal changes in hormone-sensitive and lipoprotein lipase mRNA concentrations in marmot white adipose tissue. Am J Physiol 262: R177-R181, 1992.
Yatani A, Kim S J, Kudej R K, Wang Q, Depre C, Irie K, Kranias E G, Vatner S F, and Vatner D E. Insights into cardioprotection obtained from study of cellular Ca2+ handling in myocardium of true hibernating mammals. Am J Physiol Heart Circ Physiol 286: H2219-H2228, 2004.
Yan J, Burman A, Nichols C, Alila L, Showe L C, Showe M K, Boyer B B, Barnes B M, and Marr T G. Detection of differential gene expression in brown adipose tissue of hibernating arctic ground squirrels with mouse microarrays. Physiol Genomics 25: 346-353, 2006.
Yu X X, Lewin D A, Zhong A, Brush J, Schow P W, Sherwood S W, Pan G, and Adams S H. Overexpression of the human 2-oxoglutarate carrier lowers mitochondrial membrane potential in HEK-293 cells: contrast with the unique cold-induced mitochondrial carrier CGI-69. Biochem J 353: 369-375, 2001.
Zeeberg B R, Feng W, Wang G, Wang M D, Fojo A T, Sunshine M, Narasimhan S, Kane D W, Reinhold W C, Lababidi S, Bussey K J, Riss J, Barrett J C, and Weinstein J N. GoMiner: a resource for biological interpretation of genomic and proteomic data. Genome Biology 4: R28, 2003.
Zhang W B, Zhang Z, Ni Y X, Wu Y L, and Pei G. A novel function of Goalpha: mediation of extracellular signal-regulated kinase activation by opioid receptors in neural cells. J Neurochem 86: 1213-1222, 2003.
Zhu X, Smith M A, Perry G, Wang Y, Ross A P, Zhao H W, Lamanna J C, and Drew K L. MAPKs are differentially modulated in arctic ground squirrels during hibernation. J Neurosci Res 80: 862-868, 2005.
Zimmerman U J, Wang P, Zhang X, Bogdanovich S, and Forster R. Anti-oxidative response of carbonic anhydrase III in skeletal muscle. IUBMB Life. 56: 343-347, 2004.

TABLE 1
The sequence sources of the genes on 1A and 2A arrays.

| | | | House-keeping | |
|---|---|---|---|---|
| Sequence sources | 1A array | 2A array | genes | Total |
| S. lateralis | 628 | 700 | 1 | 1,329 |
| S. parryii | 60 | 0 | 2 | 62 |
| S. tridecemlineatus | 12 | 0 | 4 | 16 |

TABLE 2
Numbers of detected and differentially expressed genes on illumina
16-sample beadchips.

| Number of | | | |
|---|---|---|---|
| genes | BAT | Liver | SKM |
| Detected | 210 | 236 | 200 |
| Differentially | 46 (22%) | 62 (26%) | 29 (15%) |
| expressed | | | |
| T > P | 34 | 25 | 16 |
| T < P | 12 | 37 | 13 |

TABLE 3
Numbers of detected and differentially expressed
genes in 96-sample illumina array matrix.

| Number of genes | BAT | Liver | Heart | Hypothalamus |
|---|---|---|---|---|
| Detected | 147 | 207 | 150 | 134 |
| Differentially | 51 (35%) | 119 (57%) | 51 (34%) | 28 (21%) |
| expressed | | | | |
| A > T | 5 | 3 | 1 | 6 |
| A < T | 23 | 36 | 33 | 0 |
| A > P | 5 | 11 | 3 | 23 |
| A < P | 7 | 78 | 7 | 1 |
| T > P | 30 | 25 | 31 | 8 |
| T < P | 11 | 43 | 8 | 2 |

TABLE 4
Numbers of genes tested in Real-time PCR experiments.

| Number of | | | | | |
|---|---|---|---|---|---|
| genes | BAT | Liver | Heart | SKM | Hypothalamus |
| Tested | 57 | 92 | 60 | 37 | 57 |
| Differentially | 24 | 56 | 28 | 17 | 28 |
| expressed | (42%) | (61%) | (47%) | (46%) | (49%) |
| A > T | 0 | 8 | 0 | 1 | 6 |
| A < T | 3 | 6 | 6 | 0 | 4 |
| A > P | 13 | 31 | 12 | 7 | 17 |
| A < P | 5 | 18 | 7 | 6 | 3 |
| T > P | 16 | 23 | 17 | 9 | 15 |
| T < P | 4 | 18 | 4 | 5 | 3 |

TABLE 5
Differential gene expression patterns in three-stage analysis.

| Expression | |
|---|---|
| Patterns | Gene symbols |
| \|0\|1\|1\| | Pdk4^{B}, Acadm^{B}, Cpt1a^{B}, Cact^{B}, Agpat3^{B}, Polr2e^{B}, |
| | Rbm3^{B}, Adcy6^{B}, Adfp^{B}, CGI-69^{B}, Map1lc3b^{B}, Glud1^{L}, |
| | Got2^{L}, Pck1^{L}, Pck2^{L}, G6pc^{L}, Fabp1^{L}, Hadhsc^{L}, |
| | Acadvl^{L}, Cpt1a^{L}, Idh2^{L}, Mdh2^{L}, Atp5a1^{L}, Rbm3^{L}, |
| | Alb^{L}, Mct12^{L}, Abcb7^{L}, Col18a1^{L}, Itih4^{L}, Pygb^{L}, |
| | Myl6^{HE}, Tmed4^{HE}, Pdhb^{HE}, Pdk4^{HE}, Cpt1a^{HE}, Fabp3^{HE}, |
| | Rbm3^{HE}, Rnh1^{HE}, Ucp2^{HE}, CGI-69^{HE}, Adfp^{HE}, |
| | Acsl3^{HY}, Rbm3^{HY}, Laptm4a^{HY}, TF^{HY}, Psma7^{HY}, |
| | Bhlhb2^{HY}, Tmbim4^{HY}, Them2^{HY}, Pdk4^{S}, Cpt1a^{S}, |
| | Rps2^{S}, Rbm3^{S}, Rnh1^{S}, Wsb2^{S}, Adfp^{S} |
| \|0\|−1\|−1\| | Scd^{B}, Prdx6^{B}, Sepp1^{B}, Arg1^{L}, Hk4^{L}, Acat2^{L}, Agpat3^{L}, |
| | Scd^{L}, Elovl6^{L}, Acacb^{L}, Sc4mol^{L}, Cat^{L}, Prdx6^{L}, |
| | Cyp1a2^{L}, Fmo5^{L}, Hspe1^{L}, Ugp2^{L}, Bckdhb^{HE}, Hpd^{HE}, |
| | Idh2^{HE}, Hsp90ab1^{HE}, Hba1^{HE}, Lonpl^{HY}, Pfkm^{S}, |
| | Gpd1^{S}, Atf4^{S} |
| \|0\|0\|1\| | Trappc5^{L}, Acadvl^{B}, Hadha^{B}, Mdh2^{B}, Got2^{HE}, Cact^{HE}, |
| | Cpt1b^{HE}, Hspe1^{HE}, Wsb2^{HE}, Cpt1b^{S}, Hadhsc^{S}, |
| | Bckdhb^{HY}, Rsu1^{HY}, Acadm^{HY}, Cox5b^{HY} |
| \|0\|−1\|0\| | Ces1^{L}, Sord^{L}, Fmo5^{B}, Igf2^{B}, Eef1a1^{HE}, Aldh2^{HE}, |
| | Alad^{HE}, Ca3^{S}, Ckm^{S}, Pkm2^{S}, Hba1^{HY} |
| \|0\|1\|0\| | Pdhb^{L}, CGI-69^{L}, Hist1h2a1^{L}, TF^{L}, Laptm4a^{L}, IDH2^{B}, |
| | Eif4b^{HE}, Hk1^{HY}, Elovl6^{HY}, Srp9^{HY} |
| \|1\|1\|0\| | Atf4^{L}, Hsp90ab1^{L}, C-myc^{L}, Adfp^{L}, Cyb5M^{L}, Gsk3b^{L}, |
| | Mdh2^{HY}, Cyp51a1^{HY}, Rnh1^{HY}, Adfp^{HY}, Abat^{HY} |
| \|−1\|0\|0\| | Hsl^{B}, Otc^{L}, Dhrs4^{L}, Ryr2^{HE}, Atp2a1^{HE}, Fabp4^{HE}, Cs^{HE}, |
| | Acacb^{HY} |
| \|−1\|0\|1\| | Cpt1b^{B}, Acaa1^{L}, Cox5b^{L}, Pdk2^{HE}, Hist1h2al^{HE}, |
| | Gnao1^{HY} |
| \|0\|0\|−1\| | Cyp51a1^{L}, Rhoc^{L}, Rps16^{B}, Lgmn^{S}, Acaa2^{HY} |
| \|−1\|1\|1\| | Apcs^{L}, Pck1^{B}, Hist1h2a1^{HY} |
| \|1\|0\|0\| | Gsk3a^{L}, Scd^{HY} |
| \|−1\|−1\|−1\| | Mgst1^{L}, Glud1^{HY} |
| \|1\|0\|−1\| | Hsp90ab1^{S} |
| \|1\|−1\|−1\| | Gsy2^{L} |
| \|1\|1\|1\| | Mdh2^{HY} |

TABLE 6
Four-stage analysis in Real-time PCR experiments.

| Number of | | | | | | |
|---|---|---|---|---|---|---|
| genes | BAT | Liver | Heart | SKM | Hypothalamus | Total |
| Tested | 57 | 92 | 60 | 37 | 57 | 303 |
| Differentially | 10 | 21 | 12 | 5 | 13 | 61 |
| expressed | | | | | | |
| EA > LA | 0 | 2 | 1 | 0 | 0 | 3 |
| EA < LA | 1 | 1 | 0 | 3 | 2 | 7 |
| EA > LT | 0 | 5 | 0 | 0 | 1 | 6 |
| EA < LT | 6 | 2 | 3 | 1 | 7 | 19 |
| LA > ET | 0 | 1 | 0 | 1 | 4 | 6 |
| LA < ET | 0 | 3 | 1 | 0 | 1 | 5 |
| ET > LT | 1 | 2 | 1 | 0 | 0 | 4 |
| ET < LT | 1 | 0 | 1 | 0 | 0 | 2 |

What is claimed is:

1. A method for treating a disease comprising:
a) administering an inhibitor or activator of a hibernation-related gene or a protein product thereof to a subject with the disease, wherein said administration is effective to treat the disease.

2. The method of claim 1, further comprising:
b) identifying the hibernation-related gene prior to administering the inhibitor or activator to a subject.

3. The method of claim 2, further comprising:
c) determining one or more inhibitors or activators of the hibernation-related gene or a protein product thereof, after identifying the hibernation-related gene and prior to administering the inhibitor or activator to a subject.

4. The method of claim 1, wherein administering the inhibitor or activator is effective to reduce the severity of at least one symptom of the disease.

5. The method of claim 1, wherein administering the inhibitor or activator is effective to eliminate at least one symptom of the disease.

6. The method of claim 1, wherein the hibernation-related gene is selected from the group consisting of Adfp (adipose differentiation-related protein), Atf4 (activating transcription factor 4), Cact (carnitine/acylcarnitine translocase), Myl6 (myosin light chain kinase 6), Ca3 (carbonic anhydrase III), Ckm (creatine kinase muscle), Rps2 (ribosomal protein S2), Lgmn (legumain), Fabpa (fatty acid binding protein, adipose), Fabph (fatty acid binding protein, heart) and Cyp51a1 (cytochrome P450 51A1).

7. The method of claim 6, wherein the hibernation-related gene is Myl6.

8. The method of claim 7, wherein the disease is selected from the group consisting of aneurysm, angina, arrhythmia, atherosclerosis, bradycardia, cardiomyopathy, stroke, congenital heart failure, congestive heart failure, myocarditis, valve disease, coronary artery disease, coronary insufficiency, dilated cardiomyopathy, hypertension, hypotension, ischemia, mitral valve prolapse, myocardial infarction, tachycardia and thromboembolism.

9. The method of claim 6, wherein the inhibitor or activator is selected from the group consisting of long-chain polyunsaturated fatty acids, VLDL, triacsin C, triacylglycerol, oleic acid, PPARα-agonists, PPARγ-ligands, troglitazone, LG268, arsenite, mitocin, cisplatin, ketoconazole, fluconazole and oxysterols.

10. The method of claim 1, wherein inhibitors or activators of at least two different hibernation-related genes are administered to the subject.

11. The method of claim 1, wherein the inhibitor is an siRNA inhibitor.

12. The method of claim 1, wherein the inhibitor or activator is a thyronamine derivative, analog, agonist or antagonist.

13. A method for treating a disease comprising:
a) identifying one or more protein products of a hibernation-related gene;
b) providing an expression vector that expresses the one or more protein products in a target cell; and
c) administering the expression vector to a subject with a disease, wherein said administration is effective to treat the disease.

14. The method of claim 13, wherein the hibernation-related gene is selected from the group consisting of Adfp, Atf4, Cact, Myl6, Ca3, Ckm, Rps2, Lgmn, Fabpa, Fabph and Cyp51a1.

15. A method for detecting or diagnosing a disease comprising
a) assaying a sample from at least one tissue of a subject for the levels of expression of one or more hibernation related genes, wherein the level of expression is indicative of the presence or absence of the disease.

16. The method of claim 15, wherein the one or more hibernation-related genes are selected from the group consisting of Adfp, Atf4, Cact, Myl6, Ca3, Ckm, Rps2, Lgmn, Fabpa, Fabph and Cyp51a1.

17. The method of claim 12, wherein the hibernation-related gene is Myl6.

18. The method of claim 17, wherein the disease is selected from the group consisting of aneurysm, arrhythmia, atherosclerosis, cardiomyopathy, stroke, congenital heart failure, congestive heart failure, myocarditis, valve disease, coronary artery disease, coronary insufficiency, dilated cardiomyopathy, ischemia and myocardial infarction and thromboembolism.

19. A method for treating a disease comprising:
administering an inhibitor or activator of the Myl6 gene or a protein product thereof to a subject with the disease, wherein said administration is effective to treat the disease.

20. The method of claim 19, wherein the disease is selected from the group consisting of aneurysm, angina, arrhythmia, atherosclerosis, bradycardia, cardiomyopathy, stroke, congenital heart failure, congestive heart failure, myocarditis, valve disease, coronary artery disease, coronary insufficiency, dilated cardiomyopathy, hypertension, hypotension, ischemia, mitral valve prolapse, myocardial infarction, tachycardia and thromboembolism.

21. A kit comprising:
at least one inhibitor or activator of a hibernation-related gene or a protein product thereof and a suitable container to contain the at least one inhibitor or activator.

22. The kit of claim 21, further comprising a means for administering the at least one inhibitor or activator to a subject with a disease.
